(19) United States
(12) Patent Application Publication
(10) Pub. No.: US 2018/0140693 A1
(43) Pub. Date: May 24, 2018

(54) Group A Streptococcus Pharmaceutical Compositions and Methods Thereof

(71) Applicant: University of Wollongong, Wollongong (AU)

(72) Inventors: Mark Walker, Balgownie (AU); Jason Cole, Cordeaux (AU); Anna Henningham, Gulgong (AU)

(73) Assignee: University of Wollongong, Wollongong (AU)

(21) Appl. No.: 15/863,105

(22) Filed: Jan. 5, 2018

Related U.S. Application Data
(63) Continuation of application No. 14/808,873, filed on Jul. 24, 2015, now abandoned.
Application No. 14/808,873 is a division of application No. 12/674,661, filed on Jun. 23, 2011, now abandoned.

(30) Foreign Application Priority Data
Aug. 24, 2007 (AU) 2007904592
Jun. 19, 2008 (AU) 2008903130

(51) Int. Cl. A61K 39/09

(52) U.S. Cl. CPC A61K 39/092; A61K 2039/575

(57) ABSTRACT

Isolated proteins and immunogenic fragments thereof, for use in the treatment and prevention of a Group A Streptococcus infection are provided. In particular, the invention provides pharmaceutical compositions and methods of prophylactic and/or therapeutic treatment of a Group A Streptococcus infection.

### CROSS REFERENCE TO RELATED APPLICATIONS AND CLAIM TO PRIORITY

This application is a continuation application of U.S. application Ser. No. 14/808,873 (filed Jul. 24, 2015), which application is a divisional application of U.S. application Ser. No. 12/674,661 (filed on Jun. 23, 2011), which application is a national stage application of International Application No. PCT/AU08//01236 (filed on Aug. 22, 2008), which claims priority to Australian Patent Application No. 2007904592 (filed on Aug. 24, 2007) and Australian Patent Application No. 2008903130 (filed on Jun. 19, 2008), which applications are herein incorporated by reference in their entireties and to which priority is claimed.

### REFERENCE TO SEQUENCE LISTING

This application includes one or more Sequence Listings pursuant to 37 C.F.R. 1.821 et seq., which are disclosed in computer-readable media (file name: 0650.0001C1_SeqList.txt, created Jul. 24, 2015, and having a size of 80,844 bytes), which file is herein incorporated by reference in its entirety.

### FIELD OF THE INVENTION

THIS invention generally relates to immunotherapy. Particularly, this invention relates to immunotherapeutic compositions, and more particularly vaccines, for the prophylactic and therapeutic treatment of Streptococcus pyogenes infection.

### BACKGROUND TO THE INVENTION

The Genus Streptococcus consists of numerous Gram-positive, non-motile, chain-forming cocci commonly found in the normal oral and bowel flora of warm-blooded animals. Streptococci are a diverse group of bacteria capable of colonizing and infecting a broad spectrum of host organisms and tissues.

Pathogenic streptococcal species fall into three broad categories: pathogenic species commonly causing infection in humans; commensal species and zoonotic species which under the right conditions cause opportunistic infection in humans. Streptococcus pyogenes (group A Streptococcus; GAS; S. pyogenes), S. agalactiae (group B Streptococcus; GBS), S. pneumoniae, and S. mutans are pathogenic streptococcal species commonly causing infection in humans.

The development of human streptococcal vaccines is challenging, facing obstacles such as the occurrence of many unique serotypes, antigenic variation within the same serotype, differences in geographical distribution of serotypes, and the production of antibodies cross-reactive with human tissue which can lead to host auto-immune disease. Streptococcal disease is a continuing worldwide problem, occurring in both developed and developing regions; thus the imperative for efficacious vaccines to prevent streptococcal disease is high. Furthermore, whilst antibiotics continue to be used to control streptococcal infection, there is increasing concern that such use, and particularly over-use, leads to the emergence of resistant strains [1-3]. Additionally, streptococci may avoid the effect of antibiotics via intracellular invasion [4].

Although there are many candidate antigens and vaccine preparations under investigation, currently, for all streptococcal species, there are only two licensed human vaccines for use against pneumococcal infection and there is no commercial vaccine available for prevention of GAS infection and disease.

GAS colonises the mucosa of the respiratory tract and skin causing, most commonly, pharyngitis and pyoderma. When S. pyogenes colonises normally sterile tissues severe invasive disease may result [5]. S. pyogenes is the etiologic agent for a range of diseases, ranging from mild infections (pharyngitis, scarlet fever, impetigo and cellulitis) to severe invasive diseases such as septicemia, streptococcal toxic shock syndrome and necrotizing fasciitis (flesh-eating disease).

It is estimated that at least 517,000 deaths per year are due to severe GAS diseases (e.g. acute rheumatic fever, rheumatic heart disease, post-streptococcal glomerulonephritis, and invasive infections). The prevalence of severe GAS disease is at least 18.1 million cases, with 1.78 million new cases each year [6].

The greatest disease burden is due to rheumatic heart disease with a prevalence of at least 15.6 million cases. These estimates suggest that, on a global scale, GAS is an important cause of morbidity and mortality, mainly in developing countries. For example, the minimum estimate of over 500,000 deaths per year places GAS among the major human pathogens, only exceeded by HIV, tuberculosis, plasmodium, pneumonia and comparable to measles, influenza Type B and Hepatitis B virus [6].

Currently there is no licensed vaccine to prevent GAS infection. Some experimental purified subunit vaccines against S. pyogenes are under development based upon M protein, C5a peptidase, SpeB, group A carbohydrate and the fibronectin binding proteins Sfb1, SOF and FBP54. GAS vaccinology has primarily focussed on the major virulence factor, the M protein. However, several factors have hampered the development of M protein-based vaccines such as the large number of unique M serotypes, the potential antigenic variation within a serotype due to the continual evolution of M protein [7], and the presence of cross-reacting epitopes which may trigger post-infective immune sequelae [8]. Furthermore, circulating GAS strains can rapidly be replaced by a new set of strains [9, 10]. As a consequence, N-terminal multivalent M protein vaccine preparations may need to be continuously reformulated in order to protect against current circulating strains.

M proteins are an attractive target for vaccine development as they are known to be a major virulence factor in GAS and have elicited protective immunity in several studies. Early studies involving vaccination of humans with crude M protein preparations followed by administration of live GAS to the pharynx effectively protected against GAS pharyngeal colonization [11-13], but one study noted an increased incidence of rheumatic fever in vaccinated versus unvaccinated control children [14]. This significantly hampered the development of whole M protein-based vaccines. Since then, a number of vaccine development studies have targeted the C-terminal repeat region peptides, conserved amongst all serotypes [15, 16], whilst other studies have focussed on polypeptides derived from the serotype specific N-terminal repeats [17-21]. One group has developed a hexavalent vaccine containing protective N-terminal M protein fragments from six serotypes. The included serotypes were selected due to a frequent association with pharyngitis and acute rheumatic fever [21]. This hybrid vaccine has been observed to generate high titre opsonising antibodies in rabbits [21] and to protect against murine mucosal challenge [22]. The vaccine was tested in phase I clinical trials and was found to produce a statistically significant increase in antibody titre for all six M protein fragments, with five of the six serotypes being opsonised by the resulting anti-sera [23]. Although this vaccine was successful in phase I clinical trials, the major shortcoming of this hexavalent vaccine is that it only offers protection against six of an estimated 120 GAS serotypes. In an attempt to broaden the protection, a multivalent vaccine containing variable amino terminal fragments of 26 different M proteins was produced using recombinant techniques [24]. Following immunisation of rabbits, type-specific antibodies raised against 25 of the 26 M protein fragments in the vaccine were detected and none of these antibodies cross-reacted with host tissue [24]. Additionally, this vaccine preparation was observed to be safe and immunogenic in phase I clinical trials [25]. Although this vaccine preparation shows promise for the prevention of GAS infection, it is likely a vaccine protective against all serotypes will be necessary for the eradication of GAS infection and disease.

Cole et al. [26] describes results of proteomic analysis of S. pyogenes and is incorporated herein by reference. In particular, the study by Cole et al. [26] characterised the cell wall and surface association of the selected proteins on in vitro grown cells. There is much debate in the literature as to whether or not this data happens to be “artefactual”. In support of this contention, see Cole et al. [26] and compare with Rodriguez-Ortega et al. [27].

### SUMMARY OF THE INVENTION

Despite intensive effort, an effective and safe commercial GAS vaccine is not available for human use. The present inventors have identified that a vaccine protective against all serotypes will be necessary for the control and/or eradication of GAS infection and disease.

The present invention arises from the unexpected finding that a number of cell wall-associated proteins from S. pyogenes can elicit a protective immune response in mice challenged with fatal doses of S. pyogenes. Moreover, these immunogenic proteins do not display a significant sequence identity to any human protein and/or do not elicit a specific immune response in a human following natural infection with S. pyogenes.

In one particular form, the invention is broadly directed to new and efficacious isolated proteins which are capable of eliciting an immune response, and in particular a protective immune response, upon administration to an animal and are therefore candidates for use in pharmaceutical compositions against diseases and/or conditions resulting from S. pyogenes infection.

In a first aspect, the invention provides a pharmaceutical composition for preventing or treating a S. pyogenes-associated disease, disorder or condition, comprising one or more isolated immunogenic proteins from S. pyogenes or a variant thereof, wherein said one or more isolated immunogenic proteins from S. pyogenes lack significant sequence identity to a human protein and/or do not elicit a specific immune response in a human following natural infection with S. pyogenes, together with a pharmaceutically-acceptable diluent, excipient or carrier.

In a second aspect, the invention provides a pharmaceutical composition for preventing or treating a S. pyogenes-associated disease, disorder or condition, comprising an isolated nucleic acid encoding one or more isolated immunogenic proteins or a variant thereof, wherein said one or more isolated immunogenic proteins from S. pyogenes lacks significant sequence identity to a human protein and/or do not elicit a specific immune response in a human following natural infection with S. pyogenes, together with a pharmaceutically-acceptable carrier, diluent or excipient.

Suitably, the one or more isolated immunogenic proteins from S. pyogenes are cell wall-associated proteins.

Preferably, the one or more isolated immunogenic proteins from S. pyogenes are selected from the group consisting of trigger factor (TF), ketopantoate reductase (KPR), arginine deiminase (ADI), ornithine carbamoyltransferase (OCTase), phosphotransacetylase (PTA), ribosome recycling factor (RRF), branched-chain-amino-acid aminotransferase (BCAT), carbamate kinase (CK), adenylate kinase (AK), elongation factor P (EF-P), high temperature requirement A serine protease (HtrA), phosphoglycerate kinase (PGK), 6-phosphofructokinase (PFK), NADP dependent glyceraldehyde 3 phosphate dehydrogenase (NADP-GAPDH) and Spy1262.

More preferably, the one or more isolated immunogenic proteins from S. pyogenes are selected from the group consisting of ADI, TF, KPR, OCTase, PTA, RRF and BCAT.

Even more preferably, the one or more isolated immunogenic proteins from S. pyogenes are selected from the group consisting of ADI, TF and KPR.

Compositions according to this aspect may be used either prophylactically or therapeutically.

In a third aspect, the invention provides an isolated immunogenic fragment of an isolated immunogenic protein from S. pyogenes or a variant thereof, wherein said isolated immunogenic protein from S. pyogenes lacks significant sequence identity to a human protein and/or does not elicit a specific immune response in a human following natural infection with S. pyogenes.

Preferably, the isolated immunogenic protein from S. pyogenes is selected from the group consisting of TF, KPR, ADI, OCTase, PTA, NADP-GAPDH, RRF, BCAT, CK, AK, EF-P, HtrA, PGK, PFK and Spy1262.

More preferably, the isolated immunogenic protein from S. pyogenes is selected from the group consisting of ADI, TF, KPR, OCTase, PTA, RRF and BCAT.

Even more preferably, the isolated immunogenic protein from S. pyogenes is selected from the group consisting of ADI, TF and KPR.

In a preferred embodiment, the isolated immunogenic fragment is a variant.

Preferably, the isolated immunogenic fragment comprises an amino acid sequence selected from the group consisting of SEQ ID NOS:2 to 4, SEQ ID NOS:9 to 22, SEQ ID NOS:26 to 30, SEQ ID NOS:33 to 37, SEQ ID NOS:43 to 46, SEQ ID NOS:48 to 50, SEQ ID NOS:52 to 54, SEQ ID NOS:60 to 62, SEQ ID NOS:67 to 74, SEQ ID NO:80, SEQ ID NO:91, SEQ ID NOS:93 to 97, SEQ ID NOS:105 to 107, SEQ ID NOS:113 to 116, SEQ ID NOS:132 to 133, SEQ ID NOS:139 to 151, SEQ ID NOS:168 to 169, SEQ ID NOS:175 to 183, SEQ ID NOS:185 to 193, SEQ ID NO:199, SEQ ID NOS:203 to 204, SEQ ID NOS:210 to 233, SEQ ID NOS:245 to 247, SEQ ID NOS:249 to 258, SEQ ID NOS:270 to 274, SEQ ID NOS:278 to 282, SEQ ID NOS:289 to 293, SEQ ID NOS:295 to 299, SEQ ID NOS:312 to 313, SEQ ID NOS:316 to 324, SEQ ID NO:327, SEQ ID NOS:334 to 338, SEQ ID NOS:341 to 345, SEQ ID NOS:349 to 350, SEQ ID NOS:352 to 358, SEQ ID NOS:361 to 362 and SEQ ID NOS:365 to 367.

In a fourth aspect, the invention provides an isolated protein comprising one or a plurality of isolated immunogenic fragments of the third aspect.

In a fifth aspect, the invention provides an isolated nucleic acid encoding the isolated immunogenic fragment of the third aspect or the isolated protein of the fourth aspect.

Suitably, the isolated nucleic acid is DNA.

In a sixth aspect, the invention provides a genetic construct comprising the isolated nucleic acid of the fifth aspect operably linked to one or more regulatory nucleotide sequences.

Preferably, the genetic construct is an expression construct.

More preferably, the expression construct is suitable for expression of a recombinant protein.

In a seventh aspect, the invention provides a host cell comprising the genetic construct of the sixth aspect.

Preferably, the host cell is of prokaryotic origin or eukaryotic origin.

More preferably, the host cell is of prokaryotic origin.

In an eighth aspect, the invention provides an antibody, or antibody fragment which binds and/or has been raised against one or more isolated immunogenic proteins from S. pyogenes which lack significant sequence identity to a human protein and/or do not elicit a specific immune response in a human following natural infection with S. pyogenes as hereinbefore described or the one or more isolated immunogenic fragments of the third aspect.

In a ninth aspect, the invention provides a pharmaceutical composition for treating or preventing a S. pyogenes-associated disease, disorder or condition comprising one or more isolated immunogenic fragments of the third aspect, the isolated protein of the fourth aspect, the isolated nucleic acid of the fifth aspect, the genetic construct of the sixth aspect or the antibody or antibody fragment of the eighth aspect, together with a pharmaceutically-acceptable diluent, excipient or carrier.

Preferably, the pharmaceutical compositions of any of the aforementioned aspects are an immunotherapeutic composition.

More preferably, the pharmaceutical composition is a vaccine.

In a tenth aspect, the invention provides a method of immunizing an animal including the step of administering a pharmaceutical composition according to any of the aforementioned aspects, to said animal to thereby induce immunity in said animal.

In an eleventh aspect, the invention provides a method of treating an animal, including the step of administering a pharmaceutical composition according to any of the aforementioned aspects, to thereby modulate an immune response in said animal to prophylactically or therapeutically treat a S. pyogenes-associated disease, disorder or condition.

In a twelfth aspect, the invention provides a method of eliciting an immune response in an animal, including the step of administering a pharmaceutical composition according to any of the aforementioned aspects, to thereby elicit an immune response in said animal.

Suitably, the methods of the aforementioned aspects facilitate induction of a humoral immune response, such as a B lymphocyte-mediated immune response, although is not limited thereto.

Preferably, the B lymphocyte-mediated immune response is a protective immune response.

Suitably, the animal is selected from the group consisting of humans, domestic livestock, laboratory animals, companion animals, performance animals, poultry and other animals of commercial importance, although without limitation thereto.

Preferably, the animal is a mammal.

More preferably, the animal is a human.

It will be appreciated that the aforementioned aspects relate to use of variants of the one or more immunogenic proteins from S. pyogenes inclusive of allelic variants.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES

In order that the invention may be readily understood and put into practical effect, preferred embodiments will now be described by way of example with reference to the accompanying figures wherein like reference numerals refer to like parts and wherein:

FIG. 1 Microscopy images showing fluorescent image on the left and transmission image on the right, post-immune sera on top, pre-immune sera on bottom. A) anti-sera from mice immunised with PBS; B) M1 anti-sera; C) ADI anti-sera; D) KPR anti-sera; E) TF anti-sera.

FIG. 2 ADI peptide membrane containing overlapping peptide spots 1-133. Panels show the response from three different 1° antisera probes: A. mouse final bleed sera, post immunisation with ADI (subcutaneous route); B. mouse pre-bleed sera, prior to immunisation; C. mouse final bleed sera, post immunisation with PBS (subcutaneous route).

FIG. 3 KPR peptide membrane containing overlapping peptide spots 1-99. Panels show the response from three different 1° antisera probes: A. mouse final bleed sera, post immunisation with KPR (subcutaneous route); B. mouse pre-bleed sera, prior to immunisation; C. mouse final bleed sera, post immunisation with PBS (subcutaneous route).

FIG. 4 TF peptide membrane containing overlapping peptide spots 1-139. Panels show the response from three different 1° antisera probes: A. mouse final bleed sera, post immunisation with TF (subcutaneous route); B. mouse pre-bleed sera, prior to immunisation; C. mouse final bleed sera, post immunisation with PBS (subcutaneous route).

FIG. 5 A. Coomassie stained gel and B. western blot analysis of ADI domains using antisera from ADI vaccinated mice showing ADI mapping using subdomain cloning. The lanes designations are as follows: Lane 1 contains full-length ADI; Lane 2 contains F1 ADI (amino acids 1-218); Lane 3 contains F2 ADI (amino acids 213-411); Lane 4 contains F3 ADI (amino acids 1-154); Lane 5 contains F4 ADI (amino acids 148-277); Lane 6 contains F5 ADI (amino acids 271-411); Lane 7 contains FBA.

FIG. 6 Serum specific IgG titre prior to intraperitoneal challenge with wild-type GAS strain pM1 (n=10) for each antigen group.

FIG. 7 Survival curves of intraperitoneal challenge experiment with wild-type GAS strain pM1. Mice were intraperitoneally challenged with a lethal dose of pM1 GAS strain (approximately 2×10⁷ cfu/mL), and the survival of the mice monitored over 10 days. * indicates a significant difference of the survival of the test antigen in comparison to mice immunised with PBS, as determined by log-rank test.

FIG. 8 The Aboriginal immune response to vaccine antigens was determined using a pool of serum (n=30) obtained from Aboriginal children living in remote communities of the NT suffering endemic GAS infection. Specific titres against vaccine antigens as determined by ELISA.

### DETAILED DESCRIPTION OF THE INVENTION

The present invention arises, in part, from the identification of new protein vaccine candidates from S. pyogenes which are particularly efficacious at eliciting a protective immune response against S. pyogenes. More particularly, when the protein candidates were injected into mice in an intraperitoneal challenge experiment, the candidates antigens were shown to substantially improve the survival of mice inoculated with a lethal dose of S. pyogenes. The unexpected nature of these results is compounded since some of the candidate proteins are not traditionally thought to be surface exposed on S. pyogenes, thus the suitability of these proteins as immunogens is not foreseen. Moreover, the inventors have addressed the long-standing problem which has plagued other GAS vaccine candidates of cross-reactivity of a S. pyogenes-protein specific antibody with human tissue. Such cross-reactivity may trigger post-infective immune sequelae.

Therefore in one particular form, the invention provides one or more isolated immunogenic proteins, or immunogenic fragments thereof, for use as a pharmaceutical composition, and in particular a vaccine, that is compatible and safe to administer to humans in order to treat a S. pyogenes-related disease, disorder or condition. More particularly, the present invention is particularly well-suited to the generation of a whole-protein based vaccine, although is not limited thereto.

It will be appreciated that S. pyogenes is the etiologic agent of numerous suppurative diseases, ranging from mild skin infections, such as pharyngitis, scarlet fever, impetigo, erysipelas and cellulitis, to severe invasive diseases such as septicemia, streptococcal toxic shock syndrome and necrotizing fasciitis. The S. pyogenes species comprises over 100 different serotypes, with the possibility of each serotype possessing more than one strain. S. pyogenes typing may use either serological techniques such as, but not limited to, Lancefield Classification and M typing or molecular typing techniques such as emm sequence typing and patterning. A number of antigens are utilised to divide S. pyogenes into serotypes including the M protein and the T protein antigen, although not limited thereto. Non-limiting examples of S. pyogenes strains include 5448, NS88.2, pM1, DSM2071, HSC5, NS192, 2036 and 20174. The genomic sequence, either in whole or in part, of several strains are available such as MGAS10394, M1 and MGAS8232. Reference is made to Table 3, which provides non-limiting examples of S. pyogenes strains which have been sequenced.

Use of the term “S. pyogenes” generally relates to serotypes and strains of S. pyogenes as are known in the art.

In one general aspect, the invention resides in a pharmaceutical composition for preventing or treating a S. pyogenes-associated disease, disorder or condition, comprising one or more isolated immunogenic proteins from S. pyogenes, wherein said one or more isolated immunogenic proteins lack significant sequence identity to a human protein and/or do not elicit a specific immune response in a human following natural infection with S. pyogenes.

In the context of the present invention, by “S. pyogenes-associated disease, disorder or condition” is meant any clinical pathology resulting from infection by S. pyogenes. Typically, S. pyogenes colonises the mucosa of the respiratory tract and skin. Diseases associated with S. pyogenes infection include pharyngitis, tonsillitis, wound and skin infections, septicemia, impetigo, vaginitis, post-partum infections, scarlet fever, cellulitis, myositis, puerperal sepsis, pericarditis, meningitis, pneumonia, septic arthritis, rheumatic fever, glomerulonephritis, streptococcal toxic shock syndrome, reactive arthritis and necrotizing fasciitis, although without limitation thereto.

In the context of the present invention, the term “immunogenic” as used herein indicates the ability or potential to generate an immune response to S. pyogenes upon administration to an animal. It is envisaged that the immune response may be either B-lymphocyte or T-lymphocyte mediated, or a combination thereof. Advantageously, by “immunogenic” is meant a B-lymphocyte response, although is not limited thereto. “Immunogenic” can also mean a neutralising antibody response.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

By “protein” is meant an amino acid polymer. The amino acids may be natural or non-natural amino acids, D- or L-amino acids as are well understood in the art.

The term “protein” includes and encompasses “peptide”, which is typically used to describe a protein having no more than fifty (50) amino acids and “polypeptide”, which is typically used to describe a protein having more than fifty (50) amino acids.

In the context of the present invention, by “lack significant sequence identity to a human protein” is meant that the level of percentage sequence identity and/or homology displayed between one or more isolated immunogenic proteins from S. pyogenes of the present invention and a protein from the human proteome is at a level such that there is minimal and/or absent a cross-reactivity between an antibody generated against the one or more immunogenic proteins from S. pyogenes of the present invention and human tissue. Preferably the level of sequence identity is less than 55%, more preferably less than 40%, 30%, 20% or 10%, even more preferably less than 8%, 5%, 4%, 3%, 2% and 1%.

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, U) or amino acid occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA). Alternatively, “percent identity” is as calculated by the BLAST algorithm at NCBI (http://www.ncbi.nlm.nih.gov/).

A “comparison window” refers to a conceptual segment of typically at least 6, 10, 12, 20 or more contiguous residues that is compared to a reference sequence. The comparison window may comprise additions or deletions (i.e., gaps) of about 20% or less (e.g. 5, 10 or 15%) as compared to the reference sequence (which does not comprise additions or deletions) for optimal alignment of the respective sequences. Optimal alignment of sequences for aligning a comparison window may be conducted by computerised implementations of algorithms (for example ECLUSTALW and BESTFIT provided by WebAngis GCG, 2D Angis, GCG and GeneDoc programs, incorporated herein by reference) or by inspection and the best alignment (i.e., resulting in the highest percentage homology over the comparison window) generated by any of the various methods selected.

By “do not elicit a specific immune response in a human following natural infection with S. pyogenes” is meant that the one or more immunogenic proteins of S. pyogenes demonstrate minimal or no specific immunoreactivity, or a response during a natural S. pyogenes infection of humans (in particular a specific immune response) which is below the threshold of detection, following a natural infection of humans with S. pyogenes. In a preferred embodiment, the one or more immunogenic proteins do not elicit a specific immune response or elicit a substantially reduced specific immune response in a human following a natural infection. Typically, the specific immune response is a humoral immune response, although without limitation thereto. It will be appreciated that in the context of a natural infection that “do not elicit a specific immune response in a human” includes that the one or more immunogenic proteins may not induce naturally occurring antibodies and in particular, cross-reactive antibodies, following natural S. pyogenes infection of humans.

In certain preferred embodiments, the one or more isolated immunogenic proteins from S. pyogenes are selected from the group consisting of trigger factor (TF; eg. GENBANK Accession No. AAM80241; SwissProt Accession No. Q879L7), ketopantoate reductase (KPR; eg. GENBANK Accession No. AAL97561; SwissProt Accession No. Q8P1F1), arginine deiminase (ADI; eg. GENBANK Accession No. AAM22954; SwissProt Accession No. Q8K5F0), ornithine carbamoyltransferase (OCTase; eg GENBANK Accession No. AAM79801; SwissProt Accession No. P65609), phosphotransacetylase (PTA; eg GENBANK Accession No. BAC64083; SwissProt Accession No. Q878S0), ribosome recycling factor (RRF; eg. GENBANK Accession No. AAL97224; SwissProt Accession No. Q8P274), branched-chain-amino-acid aminotransferase (BCAT; eg. GENBANK Accession No. AAM79233; SwissProt Accession No. Q8K7U5), NADP-GAPDH (eg GENBANK Accession No. AAM79652; SwissProt Accession No. Q8K707), carbamate kinase (CK; eg GENBANK Accession No. AAM79798; SwissProt Accession No. Q8K6Q9), adenylate kinase (AK; eg. GENBANK Accession No. AAM78668; SwissProt Accession No. P69882), elongation factor P (EF-P; eg GENBANK Accession No. AAM80181; SwissProt Accession No. P68774), high temperature requirement A serine protease (HtrA; eg GENBANK Accession No. AAK34840; SwissProt Accession No. A2RH30), phosphoglycerate kinase (PGK; eg. GENBANK Accession No. AAM80231; SwissProt Accession No. Q8K5W7), 6-phosphofructokinase (PFK; eg GENBANK Accession No. AAL97841; SwissProt Accession No. Q8P0S6) and Spy1262 (eg GENBANK Accession No. AAK34116; SwissProt Accession No. Q99ZE5).

More preferably, the one or more isolated immunogenic proteins from S. pyogenes are selected from the group consisting of ADI, TF, KPR, OCTase, PTA, RRF and BCAT.

Even more preferably, the one or more isolated immunogenic proteins from S. pyogenes are selected from the group consisting of ADI, TF and KPR.

In another general aspect, the invention provides pharmaceutical compositions for preventing or treating a S. pyogenes-associated disease, disorder or condition wherein the pharmaceutical composition comprises an isolated nucleic acid encoding one or more isolated immunogenic proteins from S. pyogenes or a variant thereof, which lacks significant sequence identity to a human protein and/or do not elicit a specific immune response in a human following natural infection with S. pyogenes, as hereinbefore described.

The term “nucleic acid” as used herein designates single- or double-stranded mRNA, RNA, cRNA, RNAi and DNA, DNA inclusive of cDNA and genomic DNA.

In other general aspects, the invention provides an isolated immunogenic fragment of an isolated immunogenic protein from S. pyogenes which lacks significant sequence identity to a human protein and/or does not elicit a specific immune response in a human following natural infection with S. pyogenes, as hereinbefore described.

A “fragment” is a segment, domain, portion or region of a protein, which constitutes less than 100% of the amino acid sequence of the protein.

In preferred embodiments of the present invention, a fragment comprises between 5 and 50 amino acids, more preferably between 6 and 40 amino acids and even more preferably 8, 9, 10, 11, 12, 13, 14, 15, 16, 17, 18, 19, 20, 21, 22, 23, 24, 25, 26, 27, 28, 29, 30, 32, 34, 36 and 38 amino acids of the isolated immunogenic protein from S. pyogenes as hereinbefore described.

It will be appreciated that a fragment of the present invention may comprise contiguous amino acids or alternatively, non-contiguous amino acids of the isolated immunogenic proteins from S. pyogenes as presently described. In preferred embodiments, the immunogenic fragment of the invention is a linear epitope.

In other preferred embodiments, the one or a plurality of isolated immunogenic fragments may reside at a particular region of protein of interest such as the N-terminus or C-terminus or alternatively, at a position of the protein of interest to enable surface exposure of the amino acids or from a position that corresponds to an active site.

Reference is made to Table 6 which provides non-limiting examples of amino acid sequences of suitable immunogenic fragments.

In relation to isolated immunogenic fragments of ADI, preferably the isolated immunogenic fragment comprises an amino acid sequence selected from the group consisting of SEQ ID NOS:2 to 4, SEQ ID NOS:9 to 22, SEQ ID NOS:26 to 30, SEQ ID NOS:33 to 37, SEQ ID NOS:43 to 46, SEQ ID NOS:48 to 50, SEQ ID NOS:52 to 54, SEQ ID NOS:60 to 62, SEQ ID NOS:67 to 74, SEQ ID NO:80, SEQ ID NO:91, SEQ ID NOS:93 to 97, SEQ ID NOS:105 to 107, SEQ ID NOS:113 to 116 and SEQ ID NOS:132 to 133. In alternative preferred embodiments, the isolated immunogenic fragment of ADI has an amino acid sequence selected from the group consisting of SEQ ID NOS:2 to 4, SEQ ID NOS:9 to 22, SEQ ID NOS:26 to 30, SEQ ID NOS:33 to 37, SEQ ID NOS:43 to 46, SEQ ID NOS:48 to 50, SEQ ID NOS:52 to 54, SEQ ID NOS:60 to 62, SEQ ID NOS:67 to 74, SEQ ID NO:80, SEQ ID NO:91, SEQ ID NOS:93 to 97, SEQ ID NOS:105 to 107, SEQ ID NOS:113 to 116 and SEQ ID NOS:132 to 133.

In relation to isolated immunogenic fragments of KPR, preferably the isolated immunogenic fragment comprises an amino acid sequence selected from the group consisting of SEQ ID NOS:139 to 151, SEQ ID NOS:168 to 169, SEQ ID NOS:175 to 183, SEQ ID NOS:185 to 193, SEQ ID NO:199, SEQ ID NOS:203 to 204 and SEQ ID NOS:210 to 233. In alternative preferred embodiments, the isolated immunogenic fragment of KPR has an amino acid sequence selected from the group consisting of SEQ ID NOS:139 to 151, SEQ ID NO:160, SEQ ID NOS:168 to 169, SEQ ID NOS:175 to 183, SEQ ID NOS:185 to 193, SEQ ID NO:199, SEQ ID NOS:203 to 204 and SEQ ID NOS:210 to 233.

In relation to isolated immunogenic fragments of TF, preferably the isolated immunogenic fragment comprises an amino acid sequence selected from the group consisting of SEQ ID NOS:245 to 247, SEQ ID NOS:249 to 258, SEQ ID NOS:270 to 274, SEQ ID NOS:278 to 282, SEQ ID NOS:289 to 293, SEQ ID NOS:295 to 299, SEQ ID NOS:312 to 313, SEQ ID NOS:316 to 324, SEQ ID NO:327, SEQ ID NOS:334 to 338, SEQ ID NOS:341 to 345, SEQ ID NOS:349 to 350, SEQ ID NOS:352 to 358, SEQ ID NOS:361 to 362 and SEQ ID NOS:365 to 367. In alternative preferred embodiments, the isolated immunogenic fragment of TF has an amino acid sequence selected from the group consisting of SEQ ID NOS:245 to 247, SEQ ID NOS:249 to 258, SEQ ID NOS:270 to 274, SEQ ID NOS:278 to 282, SEQ ID NOS:289 to 293, SEQ ID NOS:295 to 299, SEQ ID NOS:312 to 313, SEQ ID NOS:316 to 324, SEQ ID NO:327, SEQ ID NOS:334 to 338, SEQ ID NOS:341 to 345, SEQ ID NOS:349 to 350, SEQ ID NOS:352 to 358, SEQ ID NOS:361 to 362 and SEQ ID NOS:365 to 367.

In another embodiment, fragments of the other proteins described herein are also envisioned, without limitation hereto.

The invention also contemplates isolated proteins, such as polypeptides or “polytope” proteins, comprising one or a plurality of isolated immunogenic fragments of the invention, and/or an isolated nucleic acid encoding the same. For example, said fragments may be present singly or as repeats, which also includes tandemly repeated fragments. “Spacer” amino acids may also be included between one or the plurality of the immunogenic fragments present in said isolated protein.

In one embodiment, an isolated polytope protein may comprise one or a plurality of isolated immunogenic fragments of the invention.

In another embodiment, the isolated polytope protein may consist of one or a plurality of isolated immunogenic fragments of the invention.

In yet another embodiment, an isolated protein may consist essentially of one or a plurality of isolated immunogenic fragments of the invention.

By “consist essentially of” is meant in this context that the or each immunogenic fragment has one, two or no more than three amino acid residues in addition to the immunogenic fragment sequence. The additional amino acid residues may occur at one or both termini of the immunogenic fragment sequence, but is not limited thereto.

In the particular context of a polytope protein, these additional amino acid residues may be referred to as “spacer” amino acids.

The isolated immunogenic proteins, fragments and/or polytopes of the present invention may be produced by any means known in the art, including but not limited to, chemical synthesis, recombinant DNA technology and proteolytic cleavage to produce peptide fragments.

In one embodiment, isolated immunogenic proteins, fragments and/or isolated proteins and/or polytopes as hereinbefore described may be generated by chemical synthesis, inclusive of solid phase and solution phase synthesis. Such methods are well known in the art, although reference is made to examples of chemical synthesis techniques as provided in Chapter 9 of SYNTHETIC VACCINES Ed. Nicholson (Blackwell Scientific Publications) and Chapter 15 of CURRENT PROTOCOLS IN PROTEIN SCIENCE Eds. Coligan et al., (John Wiley & Sons, Inc. NY USA 1995-2008). In this regard, reference is also made to International Publication WO 99/02550 and International Publication WO 97/45444.

In another embodiment, recombinant immunogenic proteins, immunogenic fragments of the invention, and/or polytopes comprising isolated immunogenic fragments may be conveniently prepared by a person skilled in the art using standard protocols as for example described in Sambrook et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. NY USA 1995-2008), in particular Chapters 10 and 16; and CURRENT PROTOCOLS IN PROTEIN SCIENCE Eds. Coligan et al., (John Wiley & Sons, Inc. NY USA 1995-2008), in particular Chapters 1, 5 and 6.

Alternatively, isolated immunogenic fragments can be produced by digestion of a polypeptide, such as a polypeptide selected from the group consisting of TF, KPR, ADI, OCTase, PTA, NADP-GAPDH, RRF, BCAT, CK, AK, EF-P, HtrA, PGK, PFK and Spy1262, with proteinases such as endoLys-C, endoArg-C, endoGlu-C and V8-protease. The digested fragments can be purified by chromatographic techniques as are well known in the art.

In light of the foregoing, it will be appreciated that the present invention contemplates isolated nucleic acids of the isolated immunogenic proteins, fragments and isolated proteins, particularly in the form of polytopes, of the present invention.

Nucleotide sequences encoding the isolated immunogenic proteins, isolated immunogenic fragments, variants and polytopes of the invention may be readily deduced from the complete genomic nucleic acid sequence of S. pyogenes published for example in Beres et al., 2002, Proc Natl Acad Sci USA. 2002 Jul. 23; 99(15):10078-83 (NCBI Accession No. NC_004070) or under NCBI Accession No.'s NC_002737 (S. pyogenes M1 GAS) and NC_009332 (S. pyogenes strain Manfredo), although without limitation thereto.

The present invention also contemplates nucleic acids that have been modified such as by taking advantage of codon sequence redundancy. In a more particular example, codon usage may be modified to optimize expression of a nucleic acid in a particular organism or cell type.

The invention also contemplates use of modified purines (for example, inosine, methylinosine and methyladenosine) and modified pyrimidines (for example, thiouridine and methylcytosine) in nucleic acids of the invention.

It will be well appreciated by a person of skill in the art that the isolated nucleic acids of the invention can be conveniently prepared by a person of skill in the art using standard protocols such as those described in Chapter 2 and Chapter 3 of CURRENT PROTOCOLS IN MOLECULAR BIOLOGY (Eds. Ausubel et al. John Wiley & Sons NY, 1995-2008).

In one particular embodiment, an isolated nucleic acid of the present invention is operably linked to one or more regulatory nucleotide sequences in a genetic construct. A person skilled in the art will appreciate that a genetic construct is a nucleic acid comprising any one of a number of nucleotide sequence elements, the function of which depends upon the desired use of the construct. Uses range from vectors for the general manipulation and propagation of recombinant DNA to more complicated applications such as prokaryotic or eukaryotic expression of the isolated nucleic acid. Typically, although not exclusively, genetic constructs are designed for more than one application. By way of example only, a genetic construct whose intended end use is recombinant protein expression in a eukaryotic system may have incorporated nucleotide sequences for such functions as cloning and propagation in prokaryotes in addition to sequences required for expression. An important consideration when designing and preparing such genetic constructs are the required nucleotide sequences for the intended application.

In view of the foregoing, it is evident to a person of skill in the art that genetic constructs are versatile tools that can be adapted for any one of a number of purposes.

Therefore in another particular form, the invention also contemplates a genetic construct comprising one or more nucleic acid sequences encoding one or more immunogenic isolated proteins from S. pyogenes, or isolated immunogenic fragment thereof of the present invention. Methods for the generation of said genetic constructs are well known to those of skill in the art. A person of skill in the art will readily appreciate that the invention also contemplates a plurality of genetic constructs comprising one or more immunogenic proteins from S. pyogenes and/or one or a plurality of isolated immunogenic fragments of the present invention.

In one particular aspect, the invention provides a genetic construct comprising an isolated nucleic acid of the invention operably linked to one or more regulatory nucleotide sequences.

In a preferred embodiment, the genetic construct is an expression construct which is suitable for recombinant expression. Preferably, the expression construct comprises at least a promoter and in addition, one or more other regulatory nucleotide sequences which are required for manipulation, propagation and expression of recombinant DNA.

By “operably linked” is meant that said one or more other regulatory nucleotide sequence(s) is/are positioned relative to the nucleic acid(s) of the invention to initiate, regulate or otherwise control transcription thereof.

“Regulatory nucleotide sequences” present in the expression construct may include an enhancer, promoter, Shine-Dalgarno sequence, splice donor/acceptor signals, Kozak sequence, terminator and polyadenylation sequences, as are well known in the art and facilitate expression of the nucleotide sequence(s) to which they are operably linked, or facilitate expression of an encoded protein. Regulatory nucleotide sequences will generally be appropriate for the host cell or organism used for expression. Numerous types of appropriate expression constructs and suitable regulatory sequences are known in the art for a variety of host cells.

With regard to promoters, constitutive promoters (such as CMV, SV40, vaccinia, HTLV1 and human elongation factor promoters) and inducible/repressible promoters (such as tet-repressible promoters and IPTG-, metallothionine- or ecdysone-inducible promoters) are well known in the art and are contemplated by the invention. It will also be appreciated that promoters may be hybrid promoters that combine elements of more than one promoter.

Preferably, said expression construct also includes one or more selectable markers suitable for the purposes of selection of transformed bacteria (such as bla, kanR and tetR) or transformed mammalian cells (such as hygromycin, G418 and puromycin).

The expression construct may also include a fusion partner (typically provided by the expression vector) so that the recombinant protein of the invention is expressed as a fusion protein with said fusion partner. The main advantage of fusion partners is that they assist identification and/or purification of said fusion protein.

Examples of fusion partners are elsewhere herein described. Typically, fusion partners are particularly useful for isolation of a fusion protein by affinity chromatography. For the purposes of fusion polypeptide purification by affinity chromatography, relevant matrices for affinity chromatography are antibody, protein A- or G-, glutathione-, amylose-, and nickel- or cobalt-conjugated resins respectively. Many such matrices are available in “kit” form, such as the QIAexpress™ system (Qiagen) useful with (HIS6) fusion partners and the Pharmacia GST purification system. Other examples of useful fusion partners include Lumio™-tag (Invitrogen) and GST.

Suitable host cells for expression may be prokaryotic or eukaryotic, such as Escherichia coli (BL-21 and derivatives for example), yeast cells, Sf9 cells utilized with a baculovirus expression system, transgenic plants, mammalian cell lines such as lymphoblastoid cell lines and splenocytes isolated from transformed host organisms such as humans and mice, although without limitation thereto.

Expression constructs may be introduced into host cells or organisms by any of a number of well known techniques including, but not limited to, transformation by heat shock, electroporation, DEAE-Dextran transfection, microinjection, liposome-mediated transfection, calcium phosphate precipitation, protoplast fusion, microparticle bombardment, viral transformation and the like.

The invention also contemplates antibodies or antibody fragments against the immunogenic proteins and/or fragments, or variants of the invention.

Generally, antibodies or antibody fragments of the invention bind to or conjugate with the one or more immunogenic proteins from S. pyogenes or the one or more isolated immunogenic fragments of the invention.

Antibodies may be monoclonal or polyclonal, obtained for example by immunizing a suitable production animal (e.g. a mouse, rat, rabbit, sheep, chicken or goat). Serum or spleen cells may be then isolated from the immunized animal according to whether polyclonal or monoclonal antibodies are required.

Monoclonal antibodies may be produced by standard methods such as described in CURRENT PROTOCOLS IN IMMUNOLOGY (Eds. Coligan et al. John Wiley & Sons. 1995-2008) and Harlow, E. & Lane, D. Antibodies: A Laboratory Manual (Cold Spring Harbour, Cold Spring Harbour Laboratory, 1988). Such methods generally involve obtaining antibody-producing cells, such as spleen cells, from an animal immunized as described above, and fusing spleen cells with an immortalized fusion partner cell.

Recombinant antibodies are also contemplated. Selection of appropriate recombinant antibodies can be achieved by any of a number of methods including phage display, microarray or ribosome display, such as discussed in Hoogenboom, 2005, Nature Biotechnol. 23 1105, by way of example.

Also contemplated are antibody fragments that retain binding specificity such as Fab, F(ab)2, Fv, scFV and Fc fragments as well understood in the art. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilising recombinant DNA methodology.

As is also well understood in the art, in order to assist detection of antibody-antigen complexes, antibodies may be conjugated with labels including but not limited to a chromogen, a catalyst, an enzyme, a fluorophore, a chemiluminescent molecule, biotin and/or a radioisotope.

In preferred embodiments, the invention provides a pharmaceutical composition as an immunogenic composition comprising one or more isolated immunogenic proteins from S. pyogenes or one or a plurality of isolated immunogenic fragments of the invention, inclusive of variants and derivatives thereof.

As referred to hereinbefore, the present invention contemplates use of a variant of said one or more immunogenic proteins from S. pyogenes, or isolated immunogenic fragments thereof. Generally, as used herein, “variants” are the proteins of the present invention in which one, two or three amino acid residues have been deleted or replaced by different amino acids without substantial alteration to immunogenicity. It is well understood in the art that some amino acids may be changed to others with broadly similar properties without changing the immunogenicity of the epitope, so called conservative substitutions. Therefore “variants” include within their scope naturally-occurring variants such as allelic variants, homologs and artificially created mutants, for example.

Substantial changes in function are made by selecting substitutions that are less conservative and relatively fewer of these may be tolerated. Generally, the substitutions which are likely to produce the greatest changes in a protein's properties are those in which (a) a hydrophilic residue (e.g., Ser or Thr) is substituted for, or by, a hydrophobic residue (e.g., Ala, Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser) or no side chain (e.g., Gly).

In certain aspects of the present invention, protein variants share at least 80% sequence identity, preferably at least 85% or 90% sequence identity and more preferably at least 95%, 96%, 97%, 98% or 99% sequence identity with the amino acid sequences of proteins of the invention as hereinbefore described. It will be appreciated that a homolog comprises all integer values less than 100%, for example the percent value as set forth above and others.

As generally used herein, a “homolog” shares a definable nucleotide or amino acid sequence relationship with a nucleic acid or protein of the invention as the case may be.

The present invention also contemplates use of natural variants of the one or more immunogenic proteins from S. pyogenes or isolated immunogenic fragment thereof, inclusive of allelic variants but is not limited thereto.

With regard to protein variants and in particular those which are artificially-created mutants, these can be created by mutagenising a protein or by mutagenising an encoding nucleic acid, such as by random mutagenesis or site-directed mutagenesis. Examples of nucleic acid mutagenesis methods are provided in Chapter 9 of CURRENT PROTOCOLS IN MOLECULAR BIOLOGY, Ausubel et al., supra which is incorporated herein by reference.

It will be appreciated by the skilled person that site-directed mutagenesis is best performed where knowledge of the amino acid residues that contribute to biological activity is available. In many cases, this information is not available, or can only be inferred by molecular modeling approximations, for example.

In such cases, random mutagenesis is contemplated. Random mutagenesis methods include chemical modification of proteins by hydroxylamine (Ruan et al., 1997, Gene 188 35), incorporation of dNTP analogs into nucleic acids (Zaccolo et al., 1996, J. Mol. Biol. 255 589) and PCR-based random mutagenesis such as described in Stemmer, 1994, Proc. Natl. Acad. Sci. USA 91 10747 or Shafikhani et al., 1997, Biotechniques 23 304, each of which references is incorporated herein. It is also noted that PCR-based random mutagenesis kits are commercially available, such as the Diversify™ kit (Clontech).

The invention also contemplates “derivatives” of one or more isolated immunogenic proteins from S. pyogenes of the invention that lack significant homology to a human protein and/or do not elicit a specific immune response in a human following natural infection with S. pyogenes, or isolated immungenic fragments thereof, such as created by chemical modification of amino acid residues, biotinylation, conjugation with fluorochromes, addition of epitope tags (for example c-myc, haemagglutinin and FLAG tags), and fusion partners that facilitate recombinant protein expression, detection and purification (such as glutathione-S-transferase, green fluorescent protein, hexahistidine, Lumio and maltose-binding protein, although without limitation thereto).

With regard to chemical modification of amino acids, this includes but is not limited to, modification by acylation, amidination, pyridoxylation of lysine, reductive alkylation, trinitrobenzylation of amino groups with 2, 4, 6-trinitrobenzene sulphonic acid (TNBS), amide modification of carboxyl groups and sulphydryl modification by performic acid oxidation of cysteine to cysteic acid, formation of mercurial derivatives, formation of mixed disulphides with other thiol compounds, reaction with maleimide, carboxymethylation with iodoacetic acid or iodoacetamide and carbamoylation with cyanate at alkaline pH, although without limitation thereto.

In this regard, the skilled person is referred to Chapter 15 of CURRENT PROTOCOLS IN PROTEIN SCIENCE, Eds. Coligan et al. (John Wiley & Sons NY 1995-2008) for more extensive methodology relating to chemical modification of proteins.

In preferred embodiments, the invention provides a pharmaceutical compositions comprising one or more isolated immunogenic proteins from S. pyogenes or one or a plurality of isolated immunogenic fragments of the invention, inclusive of variants and derivatives thereof.

It will be appreciated that in preferred embodiments the invention provides a prophylactic and/or therapeutic treatment of S. pyogenes-associated diseases, disorders or conditions.

In a preferred embodiment, the pharmaceutical composition of the present invention is an immunogenic composition.

More preferably, the immunogenic composition is an immunotherapeutic composition.

In a particular preferred embodiment, the immunotherapeutic composition is a vaccine.

Suitable vaccines may be in the form of proteinaceous vaccines, and in particular, comprise one or more isolated immunogenic proteins from S. pyogenes or a variant thereof, which lack significant sequence identity to a human protein and/or do not elicit a specific immune response in a human following natural infection with S. pyogenes, and/or one or a plurality of isolated immunogenic fragments of the present invention.

Any suitable procedure is contemplated for producing vaccine compositions. Exemplary procedures include, for example, those described in New Generation Vaccines (1997, Levine et al., Marcel Dekker, Inc. New York, Basel, Hong Kong), which is incorporated herein by reference.

Alternatively, a vaccine may be in the form of a nucleic acid vaccine and in particular, a DNA vaccine. A useful reference describing DNA vaccinology is DNA Vaccines, Methods and Protocols, Second Edition (Volume 127 of Methods in Molecular Medicine series, Humana Press, 2006) and is incorporated herein by reference.

### Methods of Immunisation and Treatment

One particular broad application of the present invention is provision of methods of treating S. pyogenes using the pharmaceutical compositions of the present invention.

Accordingly, the invention provides a method of immunizing an animal including the step of administering a pharmaceutical composition of the present invention to an animal to thereby induce immunity in said animal.

The invention also provides a method of treating an animal to thereby modulate an immune response in said animal to prophylactically or therapeutically treat a S. pyogenes-associated disease, disorder or condition.

Such compositions may be delivered for the purposes of generating immunity, preferably protective immunity, to S. pyogenes upon administration to a host, although without limitation thereto.

It will also be appreciated that the antibodies of the present invention may be useful for passive immunisation against S. pyogenes infection.

The pharmaceutical compositions of the present invention may further comprise a pharmaceutically-acceptable carrier, diluent or excipient.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991) which is incorporated herein by reference.

It will be appreciated by the foregoing that the immunotherapeutic composition and/or vaccine of the invention may include an “immunologically-acceptable carrier, diluent or excipient”.

Useful carriers are well known in the art and include for example: thyroglobulin; albumins such as human serum albumin; toxins, toxoids or any mutant crossreactive material (CRM) of the toxin from tetanus, diphtheria, pertussis, Pseudomonas, E. coli, Staphylococcus, and Streptococcus; polyamino acids such as poly(lysine:glutamic acid); influenza; Rotavirus VP6, Parvovirus VP1 and VP2; hepatitis B virus core protein; hepatitis B virus recombinant vaccine and the like. Alternatively, a fragment or epitope of a carrier protein or other immunogenic protein may be used. For example, a T cell epitope of a bacterial toxin, toxoid or CRM may be used. In this regard, reference may be made to U.S. Pat. No. 5,785,973 which is incorporated herein by reference.

The “immunologically-acceptable carrier, diluent or excipient” includes within its scope water, bicarbonate buffer, phosphate buffered saline or saline and/or an adjuvant as is well known in the art. As will be understood in the art, an “adjuvant” means a composition comprised of one or more substances that enhances the immunogenicity and efficacy of a vaccine composition. Non-limiting examples of suitable adjuvants include squalane and squalene (or other oils of animal origin); block copolymers; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); Bordetella pertussis antigens; tetanus toxoid; diphtheria toxoid; surface active substances such as hexadecylamine, octadecylamine, octadecyl amino acid esters, lysolecithin, dimethyldioctadecylammonium bromide, N,N-dicoctadecyl-N′, N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminum phosphate, aluminum hydroxide or alum; interleukins such as interleukin 2 and interleukin 12; monokines such as interleukin 1; tumour necrosis factor; interferons such as gamma interferon; combinations such as saponin-aluminium hydroxide or Quil-A aluminium hydroxide; liposomes; ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran alone or with aluminium phosphate; carboxypolymethylene such as Carbopol′ EMA; acrylic copolymer emulsions such as Neocryl A640 (e.g. U.S. Pat. No. 5,047,238); water in oil emulsifiers such as Montanide ISA 720; poliovirus, vaccinia or animal poxvirus proteins; or mixtures thereof.

With regard to subunit vaccines, an example of such a vaccine may be formulated with ISCOMs, such as described in International Publication WO97/45444.

An example of a vaccine in the form of a water-in-oil formulation includes Montanide ISA 720, such as described in International Publication WO97/45444.

In a preferred embodiment, the immunotherapeutic composition of the present invention comprising the one or more isolated immunogenic proteins from S. pyogenes that lack significant sequence identity to a human protein and/or do not elicit a specific immune response in a human following natural infection with S. pyogenes, is in the form of purified full-length protein that has been adjuvanted with Alum.

In alternative embodiments, the immunogenic proteins and/or peptides of the present invention could be used as a vaccine in the purified form, fused to immunogenic carrier proteins, or expressed by live vaccine delivery systems including attenuated viruses, virus-like particles or live attenuated bacteria.

Compositions and vaccines of the invention may be administered to humans in the form of attenuated or inactivated bacteria that may be induced to express one or more isolated immunogenic proteins or immunogenic fragments of the present invention. Non-limiting examples of attenuated bacteria include Salmonella species, for example Salmonella enterica var. Typhimurium or Salmonella typhi. Alternatively, other enteric pathogens such as Shigella species or E. coli may be used in attenuated form. Attenuated Salmonella strains have been constructed by inactivating genes in the aromatic amino acid biosynthetic pathway (Alderton et al., Avian Diseases 35 435), by introducing mutations into two genes in the aromatic amino acid biosynthetic pathway (such as described in U.S. Pat. No. 5,770,214) or in other genes such as htrA (such as described in U.S. Pat. No. 5,980,907) or in genes encoding outer membrane proteins, such as ompR (such as described in U.S. Pat. No. 5,851,519).

Expression of the proteins, peptides or fusion proteins containing transport or immunogenic functions and could result in production of the immunogenic protein or peptide in the cytoplasm, cell wall, exposed on the cell surface or produced in a secreted form.

Any safe route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular and transdermal administration may be employed.

Dosage forms include tablets, dispersions, suspensions, injections, solutions, syrups, troches, capsules, nasal sprays, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Pharmaceutical compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

So that the invention may be readily understood and put into practical effect, the following non-limiting Examples are provided.

### EXAMPLES

### Example 1

Table 1 is a listing of the protein and gene designations used in this study whilst Table 9 provides bioinformatic databank details for these proteins. Table 2 is a listing of the GAS strains, their emm sequence type and clinical origin, that were used in this study.

### Percent Homology Amongst Sequenced Gas Genomes

Conservation of the genes encoding the proteins of interest amongst a high frequency of or all serotypes is crucial when considering putative vaccine candidates. Currently there are 12 complete and one partial GAS sequenced genome, which were selected for whole genome sequencing due to a high rate of association of those serotypes with infection in the US.

Please see Table 3 for the percent identity amongst the 13 sequenced GAS genomes. The genes encoding the proteins of interest share significant homology amongst the sequenced GAS genomes.

### Presence of Gene Encoding Proteins of Interest in Different Strains

A number of representative group A streptococcal strains were screened via the polymerase chain reaction (PCR) for the presence of the genes encoding the genes of interest. When considering a protein vaccine candidate, it is important to confirm that the gene encoding the candidate protein is highly conserved amongst strains, and thus has the potential to confer protection amongst many or all circulating strains. Table 2 lists the characteristics of the strains used in the screening experiments.

Table 4 shows the frequency of the genes of interest in a selection of representative group A streptococcal strains. The results of the PCR gene screening experiments showed that the genes encoding each of the proteins were present in all representative group A streptococcal strains utilised in this study (Table 2). Given that the genes encoding the proteins were present in each of the representative GAS strains examined, it follows that these antigens should offer broad serotype-independent protection against GAS.

### Detection of Proteins of Interest in Cell Surface Extracts

A western blot is a method to detect the presence of a specific protein in a tissue homogenate or extract. In this case, GAS cell-surface extracts were obtained using the enzyme mutanolysin. These extracts were subjected to 1D electrophoresis and transferred to a nitrocellulose membrane for detection of proteins of interest using each specific anti-sera. In addition to detecting the presence of the genes via PCR, the detection of the proteins in cell extracts is important for confirmation that the protein is actually being expressed in that strain under those given conditions. Table 5 provides a summary of the presence of proteins of interest detected in mutanolysin cell-surface extracts confirming expression of the proteins across strains of different serotype. The detection of the expressed product in these GAS isolates suggests that the target immunogenic proteins are broadly expressed in GAS strains, indicating that broad cross-serotype protection will be stimulated upon vaccination.

### Example 2

The candidate antigens from S. pyogenes were expressed and purified as recombinant proteins, then used to generate anti-sera. For the antigens ADI, TF and KPR, the anti-sera generated was tested in immunofluorescence microscopy for the reactivity against the surface of GAS. The reactivity of this anti-serum against peptide-spotted membranes based on the ADI, TF and KPR amino acid sequences was also examined. Additionally, the reactivity of the ADI anti-serum against recombinant ADI domains was also examined using western blots.

### Immunofluorescent Microscopy

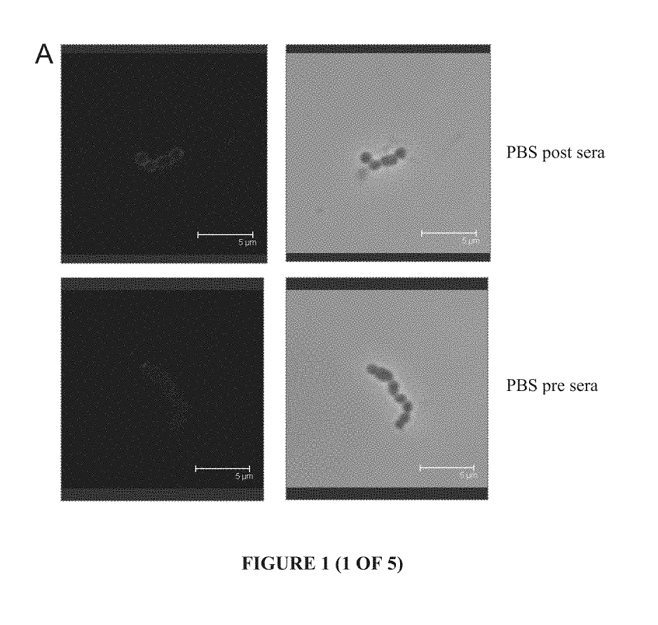

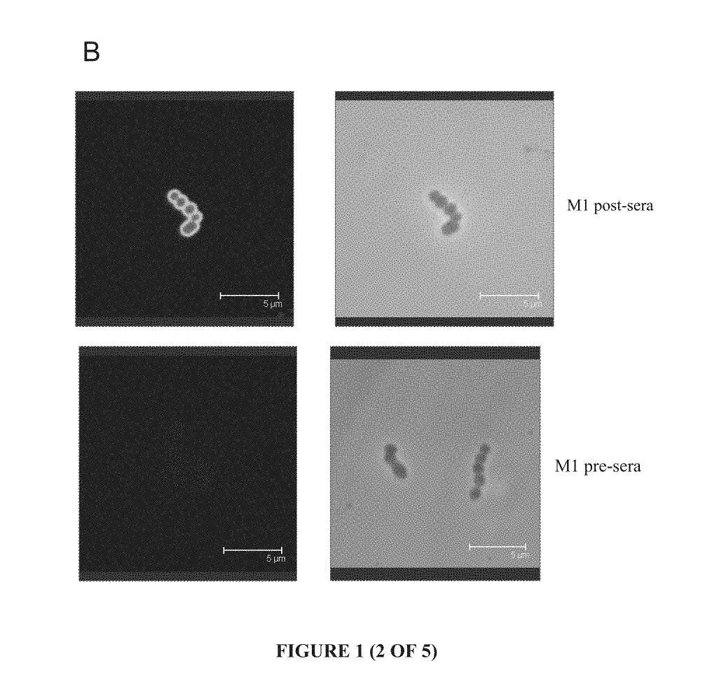

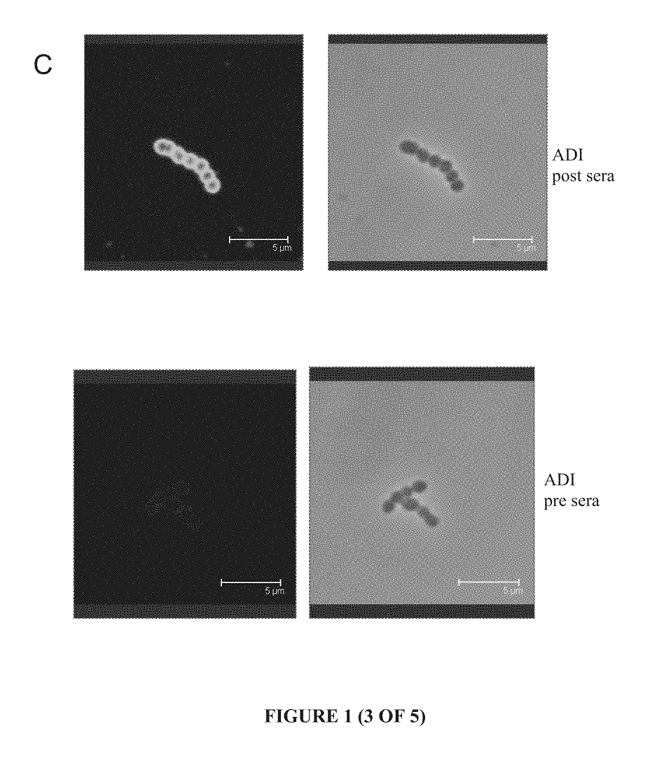

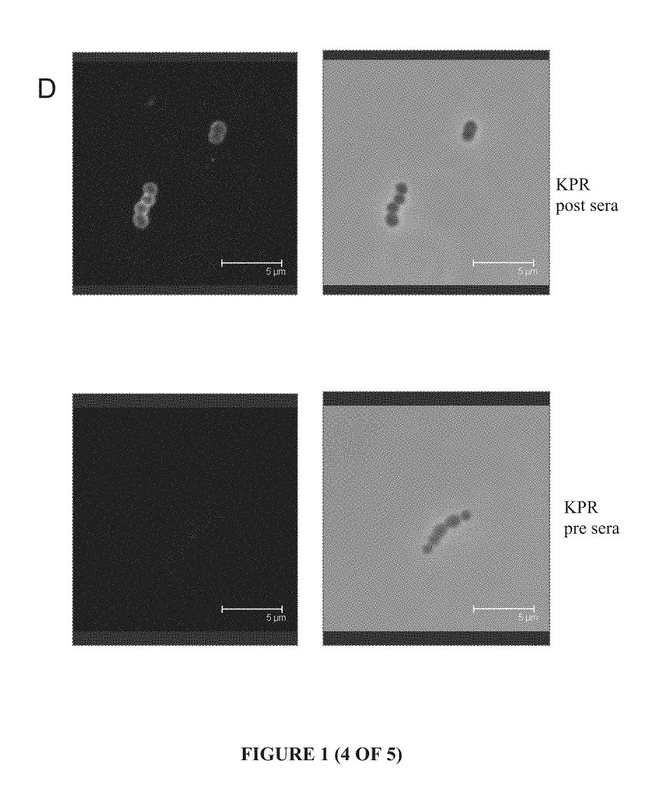

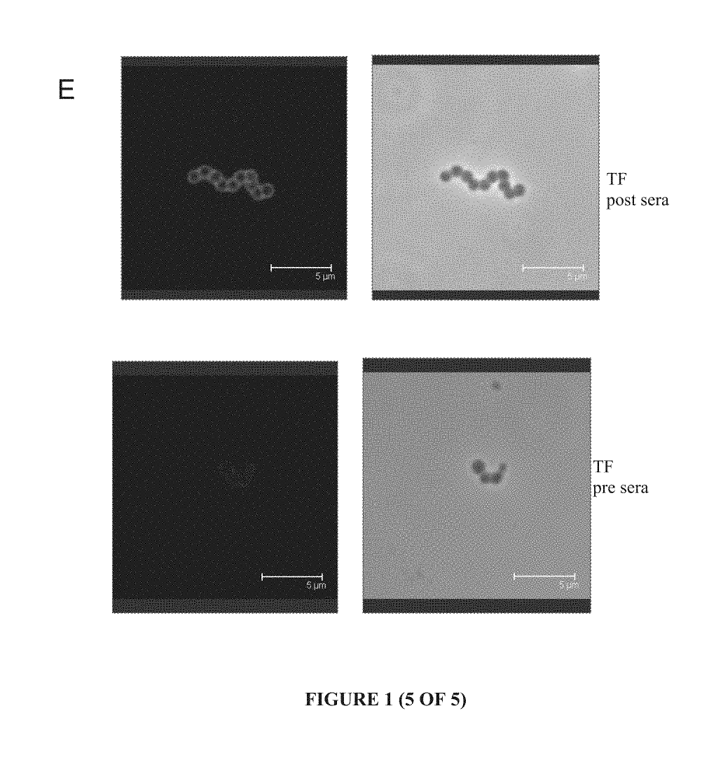

Immunofluorescence (IF) microscopy, performed with a confocal microscope, used murine anti-serum raised against the proteins of interest to allow the detection and visualisation of the proteins on the surface of whole GAS cells. The known cell-surface M protein is utilised as a positive control, and serum obtained from mice immunised with PBS is used as a baseline sample. FIG. 1 are a selection of representative images resulting from the IF analysis. The post-immune sera for the three proteins was observed to fluoresce on the surface of the cell in comparison to the pre-immune sera and sera from mice immunised with PBS (FIG. 1). These results indicate that these proteins are localised on the cell-surface, and as such may be presented to the host immune system during human infection.

### Epitope Mapping of ADI, KPR and TF

The overlapping peptide SPOT-membranes are constructed from acid-stable AC-S01 type amino-PEGylated membranes (AIMS-Scientific-Products GmbH, Braunschweig, Germany) providing a solid-phase on which short 15-mer synthetic peptides are assembled at discrete spots. The first spot corresponds to the first 15 amino acids at the N-terminus of the protein. In the second spot, the sequence slides 3 amino acids downstream of the N-terminus (in effect, repeating the last 12 amino acids from spot 1, and adding 3 at the end). This process repeats itself until the C-terminus of the protein is reached. In this investigation, the proteins used to construct the membranes were ADI, KPR, and TF. Once assembled, the membranes were probed with post-immune sera from mice vaccinated with the specific antigen (ADI, KPR, or TF). This involved a 3 hour incubation with the 1° antisera (diluted 1:100), followed by a 1.5 hour incubation with the 2° antibody (goat anti-mouse IgG conjugated to alkaline phosphatase; Sigma; 1:2000 dilution). The membranes were then developed and scanned using a densitometer, before all bound antibodies were stripped. This protocol was then repeated using pre-immune mouse sera, and PBS vaccinated post-immune mouse sera as controls. By comparing the spot signals from each treatment, the antibody-binding epitopes were identified.

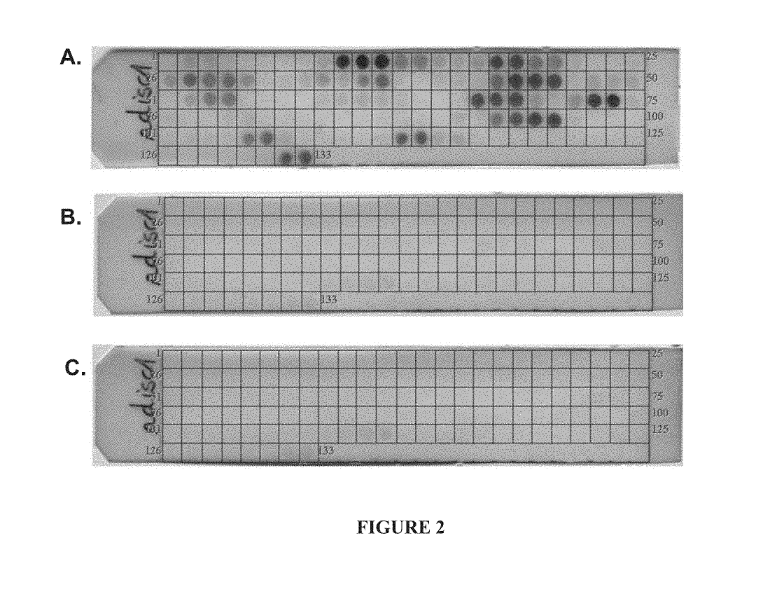

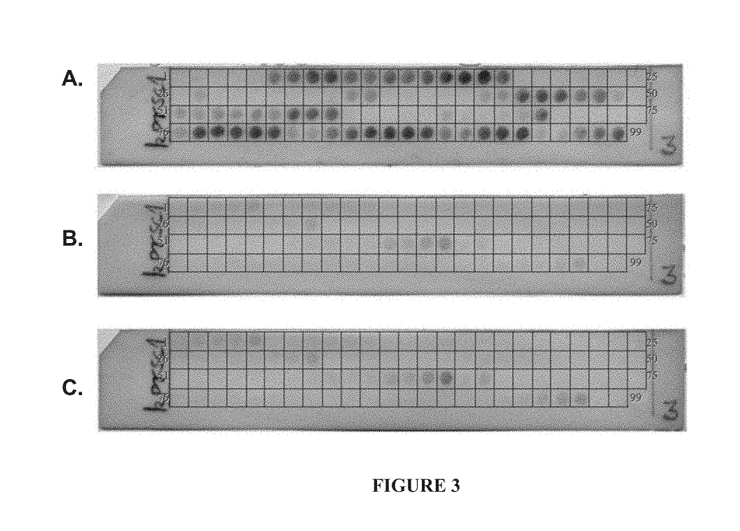

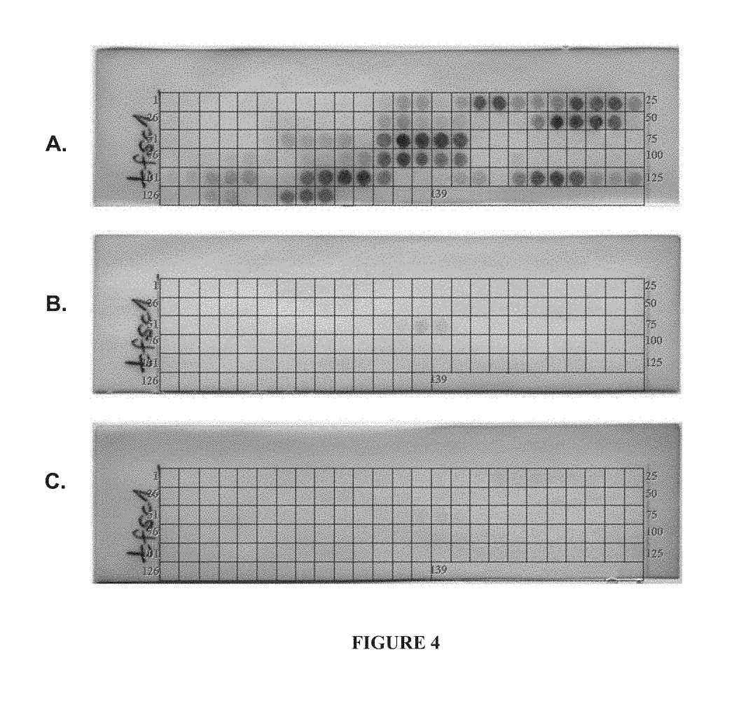

The results are shown in FIGS. 2 to 4 and Table 6. The results obtained using the post-immune mouse sera clearly show discrete regions of the membrane presenting a strong colorimetric response. The nature of the response in these regions (i.e. faint colour at first, strengthening in intensity towards the middle, and then fading again at the tail-end) suggest linear epitopes. Spots with a faint response at either end of the region would contain peptides with only a few amino acids belonging to the epitope, whereas spots with strong responses would contain the bulk of the epitope. Both controls (mouse pre-bleed sera and mouse final bleed sera, post immunisation with PBS) show very faint responses in only a few spots per membrane that could be due to artefacts within the mouse blood (i.e. non-specific interaction with other mouse immunoglobulins). What is important, however, is that these responses are consistent between the controls, and appear to be in different regions than those presenting response in the post-immune probes (i.e. only the spot with the strongest response in the KPR controls—65—shows a response in the post-immune probe, and this is noticeably fainter).

### Mapping of Epitopes of ADI by Subcloning Domains

Plasmid constructs were transformed into One Shot® BL21 Star™ (DE3) chemically competent E. coli (Invitrogen, USA) according to the manufacturer's instructions. Transformed cells were grown in 1 L cultures at 37° C. until OD₆₀₀ of 0.6 was reached. Expression was induced by addition of 1 mM IPTG and incubation for 4 hours at 37° C. Cells were harvested by centrifugation at 5,000×g for 10 min at 4° C. (JLA-10.500 rotor, J2-MC centrifuge, Beckman, USA). Recombinant proteins were purified on Ni-NTA resin (Qiagen, Australia), exploiting the Ni affinity of the 6-His tag, under denaturing conditions as outlined in the QIAexpressionist manual, 2003 (Qiagen, Australia). Purified recombinant proteins were treated prior to analysis with either 2× or 5× cracking buffer (Qiagen, Australia) and boiled for 10 min. They were then analysed by sodium dodecyl sulfate polyacrylamide gel electrophoresis (SDS-PAGE) using a Mini PROTEAN® Cell System (BioRad, USA) according to the method of Laemmli (1970). Gels to be directly visualised were stained in Coomassie blue staining solution (0.2% (w/v) Coomassie blue R-250, 40% (v/v) methanol, 10% (v/v) glacial acetic acid, 50% (v/v) dH₂O) by microwaving on high for 1 min and then shaking for 1 hour on an orbital shaker. Gels were subsequently de-stained in rapid de-stain solution (40% (v/v) methanol, 10% (v/v) glacial acetic acid, 50% (v/v) dH₂O) by microwaving on high for 1 min and shaking for 1 hour. Rapid de-stain solution was then replaced with final de-stain solution (10% (v/v) glacial acetic acid, 4% (v/v) glycerol, 86% (v/v) dH₂O) and left gently shaking overnight. Proteins on gels that had not been stained were transferred to a nitrocellulose membrane at 30 V overnight at 4° C. using the Mini Trans-Blot® (BioRad, USA). Post-transfer, membranes were blocked in a solution of 5% (w/v) skim milk (Difco) in PBS (137 mM NaCl, 2.7 mM KCl, 7.9 mM Na₂HPO₄, 1.5 mM KH₂PO₄; pH 7.4) overnight at 4° C. After 2×5 min washes with PBST (137 mM NaCl, 2.7 mM KCl, 7.9 mM Na₂HPO₄, 1.5 mM KH₂PO₄, 0.05% (v/v) Tween-20; pH 7.4), the membranes were incubated for 1 h with mouse anti-ADI protective antisera diluted 1:10,000 in 0.5% (w/v) skim milk in PBS. Following five washes for 6 min each with PBST and a 30 min blocking in 5% (w/v) skim milk in PBS, the membranes were incubated for 1 h with a 1:10,000 dilution of goat anti-mouse IgG HRP conjugate (Kirkegaard & Perry Laboratories, USA). Excess secondary antibody was removed by three PBST washes for 5 min each followed by three 5 min PBS washes. Blots were developed in a solution of 100 mM Tris-HCl (pH 7.6) containing 1.4 mM diaminobenzidine and 0.06% (v/v) hydrogen peroxide.

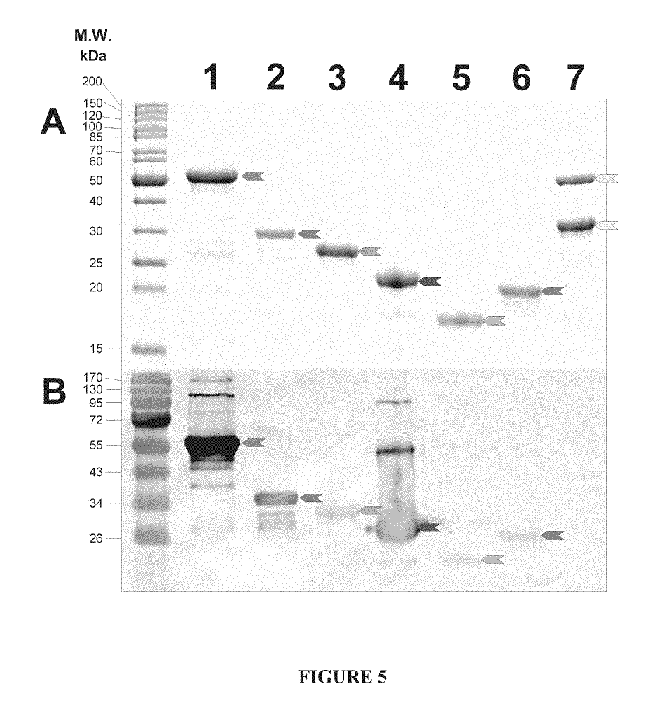

FIG. 5 shows the results of this experiment. After probing with protective ADI anti-sera, bands are visible on the blot for each of the ADI fragments (F1 ADI, amino acids 1-218; F2 ADI, amino acids 213-411; F3 ADI, amino acids 1-154; F4 ADI, amino acids 148-277; F5 ADI, amino acids 271-411). This suggests that antigenic epitopes within ADI are spread throughout the proteins structure. There does appear to be a higher level of response in F1 ADI and F3 ADI than the other fragments, therefore epitopes may be more prevalent towards the N-terminus. FBA contains the same 6-His and Lumio™ tags as ADI and the ADI fragments, and was included to investigate the presence of any background response due to antibodies in the antisera reactive with these elements. As there was no response for FBA on the blot, the 6-His and Lumio™ tags appear not to cause any interference. Full length ADI was the positive control.

### Example 3

This study is assessing the protective efficacy of a number of different putative cell-surface proteins as GAS vaccine candidates. The present inventors have expressed and purified the recombinant proteins of interest, undertaken an intraperitoneal murine challenge experiment challenging with the wild-type GAS strain pM1 and a subcutaneous murine challenge experiment challenging with the hyperinvasive covS mutant GAS strain 5448AP.

### ELISA

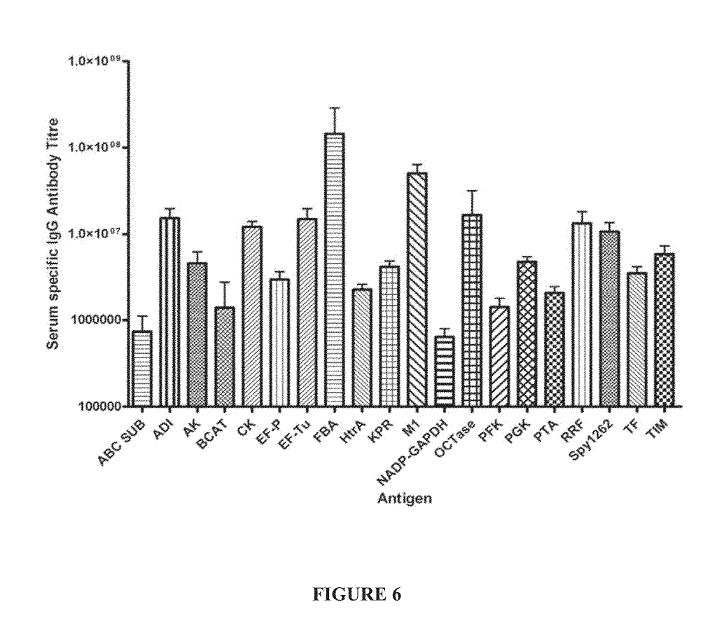

To determine the immunogenicity of the antigens, following immunisation the levels of serum-specific IgG antibody directed against the recombinant proteins were measured. Balb/c mice were immunized on day 0, 21 and 28 with 10 μg protein in a 50 μL volume via the subcutaneous route. The primary immunization contained protein emulsified 1:1 with Freunds Complete Adjuvant whilst the booster immunizations consisted of protein in PBS. The bleed was performed on day 41. To standardise amongst all groups, serum obtained from mice immunised with PBS was also tested for reactivity against the recombinant proteins (as it was not expected to react, this served as the background). The titres shown in FIG. 6 are for the final bleed only performed prior to challenge, representative of the titres in the mice at the time of challenge.

### Intraperitoneal Challenge Data

An intraperitoneal challenge experiment was selected as an en bloc approach to initially screen the immunogenicity and protective efficacy of the selected antigens. Following intraperitoneal challenge with a lethal dose of wild type GAS strain pM1 (serotype M1, SpeB-positive, covRS wild-type genotype), the Balb/c mice were monitored for a period of 10 days and survival curves generated. Using the log-rank test, statistical significance between the PBS (negative control) and the test antigens was assessed.

Balb/c mice were immunized as previously described.

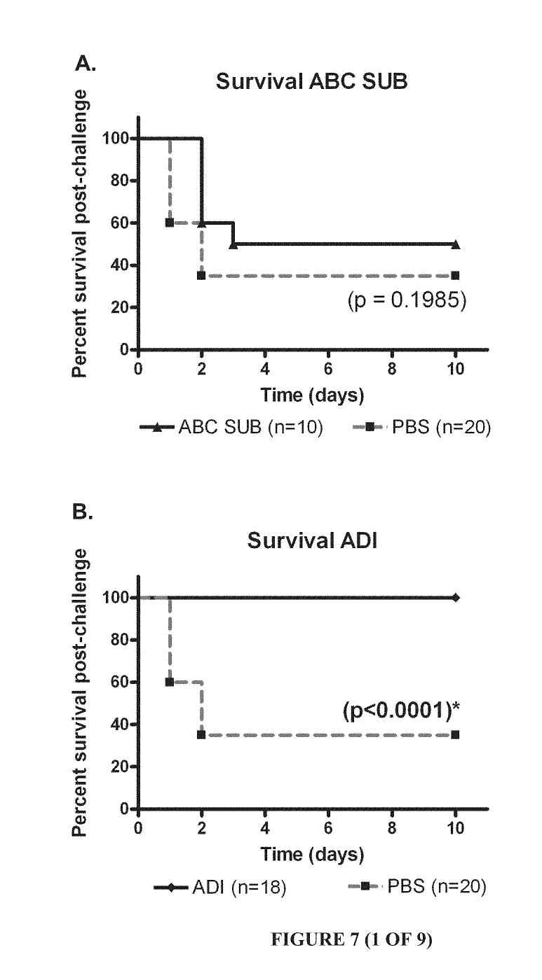

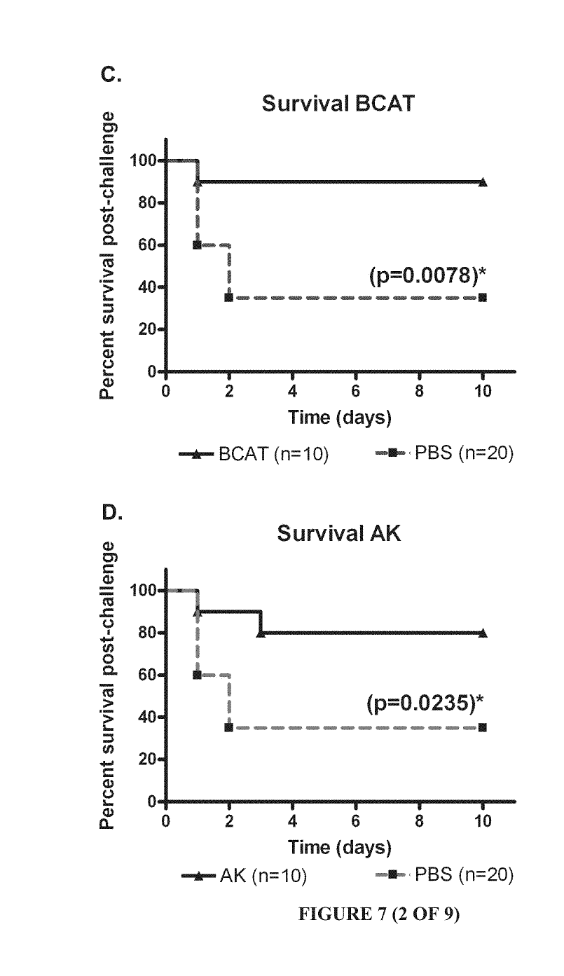

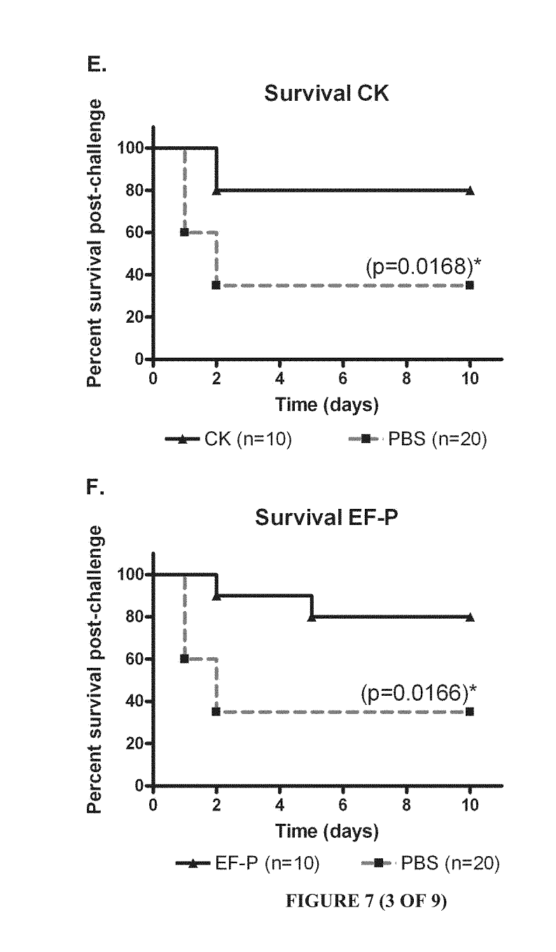

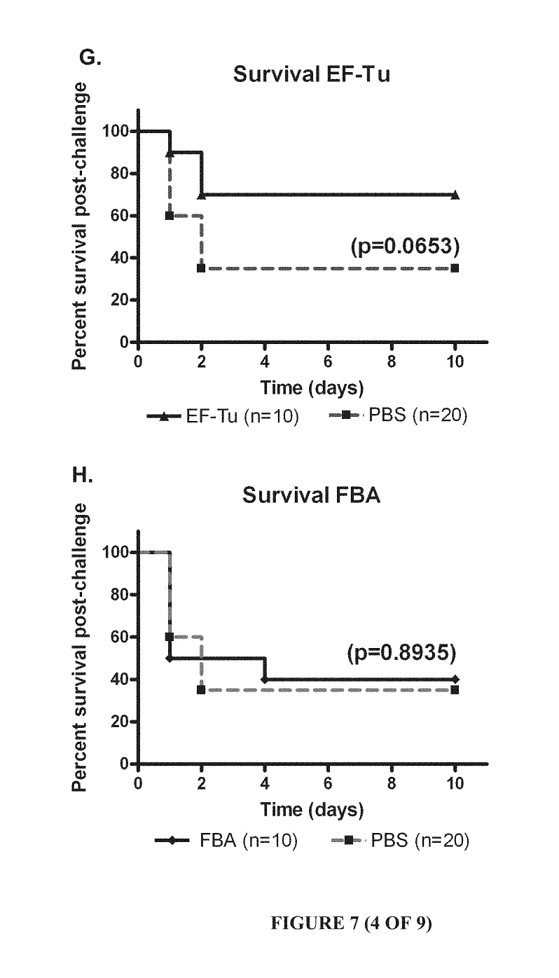

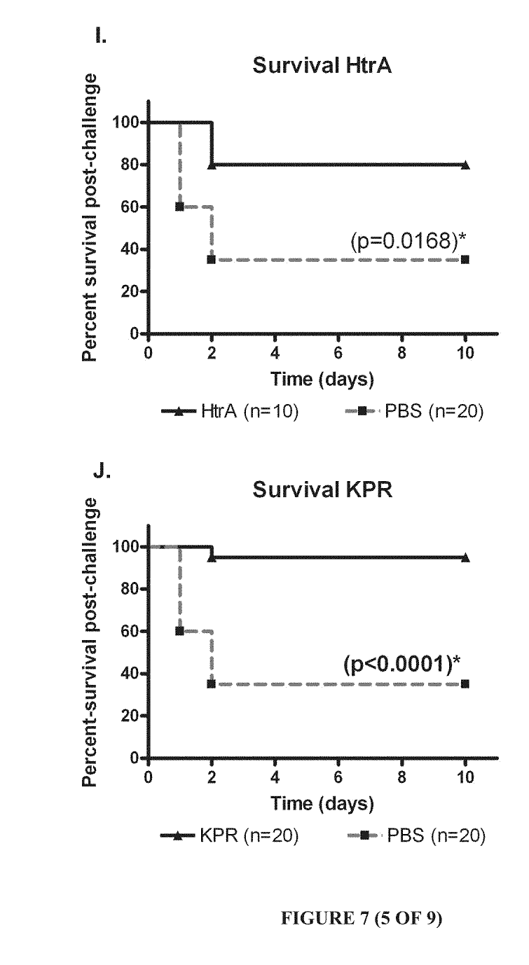

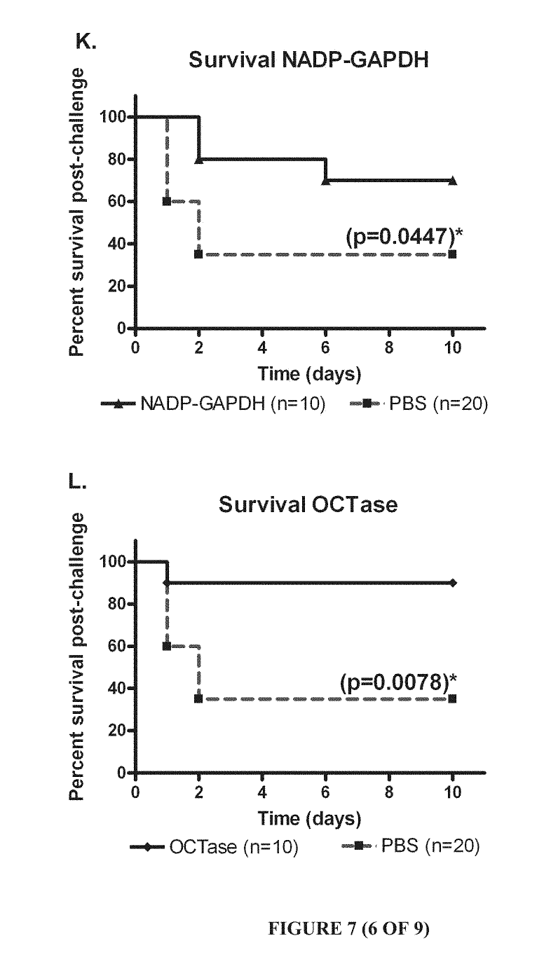

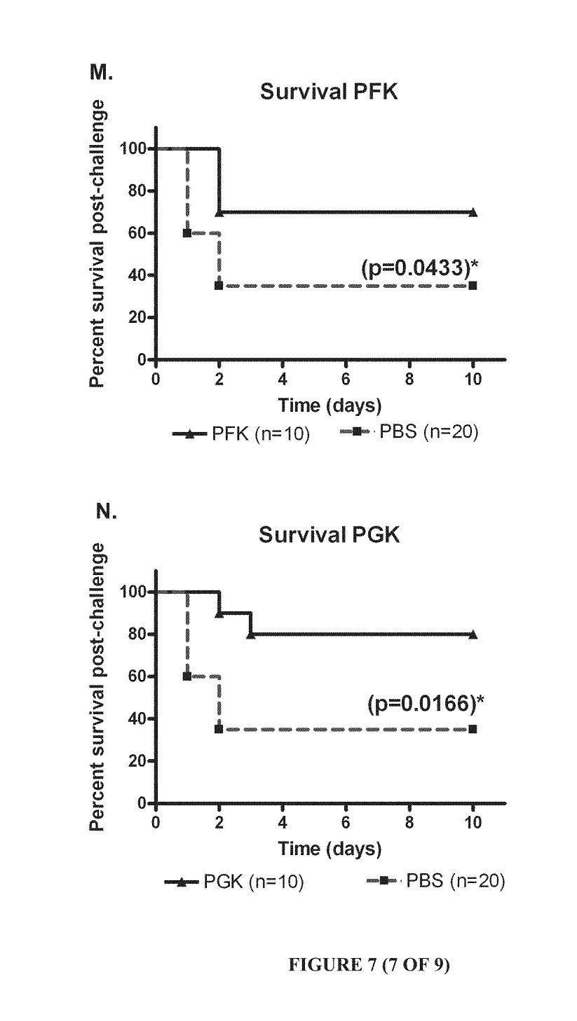

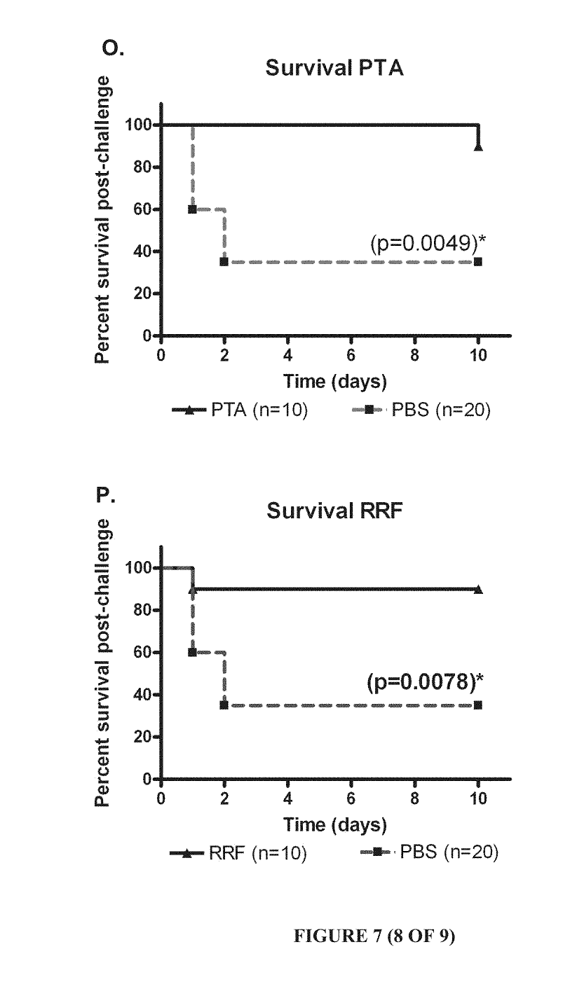

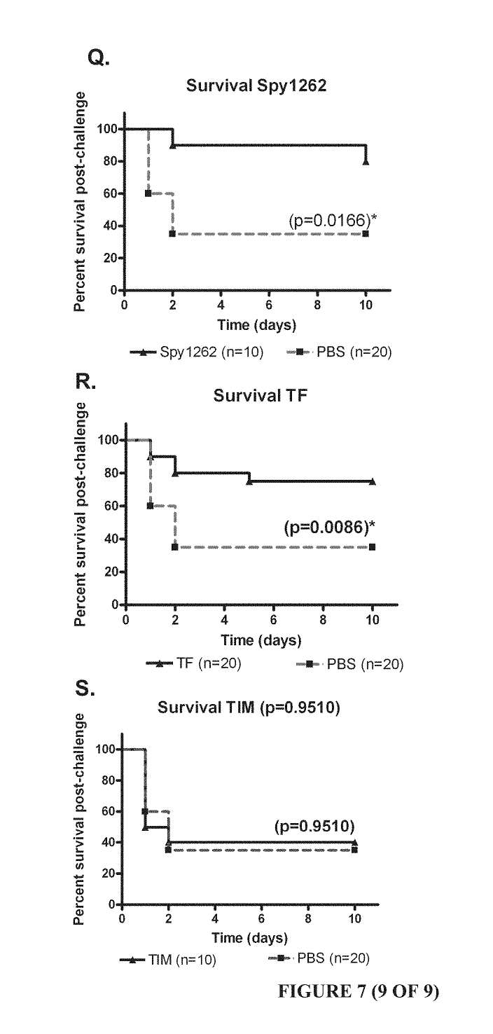

FIG. 7 demonstrates that immunization with ADI, TF, KPR, OCTase, PTA, RRF and BCAT show significant levels of protection (p<0.01) using this immunization regime, with lethal intraperitoneal challenge using the wild type strain pM1 (serotype M1, SpeB-positive, covRS wild type genotype). Additionally, CK, AK, EF-P, HtrA, PGK, PFK, NADP-GAPDH and Spy1262 show partial protection in this challenge system (p<0.05).

Table 7 provides non-limiting examples where an antigen that may have been found to protect against challenge in other bacterial species were not found to be protective against GAS challenge in this study. Of note, the GAS protein FBA was found not to protect against GAS challenge, despite the previous observation that FBA provided partial protection against Streptococcus pneumoniae challenge [28]. This observation highlights the fact that protection observed in other streptococcal species in not necessarily indicative of protection against GAS challenge.

### Subcutaneous Challenge Data

Apart from intraperitoneal challenge with the wild-type GAS M1 serotype strain pM1, a parallel set of experiments was undertaken using a different GAS challenge strain, immunisation route and challenge route. Each set of experiments contained groups of 10 mice, and was undertaken at least twice.

The GAS challenge strain used in this set of experiments is the M1T1 hyperinvasive isolate 5448AP, which is described in the following publication (Walker et al. 2007 Nature Medicine 13: 981-985). This strain is associated with invasive disease of humans, and show higher levels of virulence in comparison to wild-type strains in a subcutaneous mouse challenge model. The cause of this hypervirulence is a result of a mutation in the covS control of virulence regulatory gene. It is known that strains carrying such mutations are more frequently isolated from human invasive disease and that this type of mutation results in gene expression changes to approximately 15% of the GAS genome. Of the many changes in gene expression, it is known that SpeB protease is switched off and capsule expression is upregulated (Sumby et al, 2006 PLoS Pathogens 2: 41-49). These changes in gene expression may result in increased GAS resistance to human neutrophils (Walker et al. 2007 Nature Medicine 13: 981-985). Capsule expression increases may also mask GAS surface antigens to the immune system, making vaccine development more difficult. However, this mutant phenotype, though more resistant to human neutrophils may not be able to effectively colonise the human host due to changes in gene expression or regulation caused by the covS mutation.

The immunisation route used in these experiments was intraperitoneal injection of 10 μg of each antigen, combined with Freund's complete adjuvant on day 0, and booster immunisations on days 21 and 28 where protein was resuspended in PBS. On day 56, mice were challenged with approximately 1×10*8 of 5448AP colony forming units (doses from individual challenge experiments ranged from 1×10*8 to 2×10*8 colony forming units) by subcutaneous delivery and mouse survival monitored over a 10 day period. Using the log-rank test, statisical significance between mice immunised with PBS (negative control) and mice immunised with test antigens was assessed.

To summarise the results obtained from these series of experiments, in comparison to PBS negative control groups (33% survival), purified M1 protein was able to protect 91% of mice in this vaccination group over the 10 day course of the experiment (p<0001). Elevated levels of protection was observed for mice groups immunised with KPR (49% survival; p=0.081), EF-P (60% survival; p=0.069) and ADI (48% survival; p=0.075). All other single antigens provided minimal or no protection. However, two separate combinations of two antigens ADI+OCTase (60% survival; p=0.037) and ADI+TF (67% survival; p=0.007) were found to provide significant protection from invasive infection. Three other combinations of antigens examined did not provide protection.

These data suggest that the correct combination of GAS antigens described here have potential to not only protect against wild-type bacteria but also the hypervirulent form of GAS containing mutations in covR/S.

### Example 4

The identification of proteins which are antigenically conserved and not cross-reactive with host tissue is an important issue for the development of GAS vaccines. We have therefore examined the reactivity of serum taken from children living in an area where GAS infections are endemic, and also examined the percentage amino acid sequence identity of the candidate vaccines described here with the human proteome.

### Detection of Immune Response Against Proteins in Human Endemic Sera Using ELISA

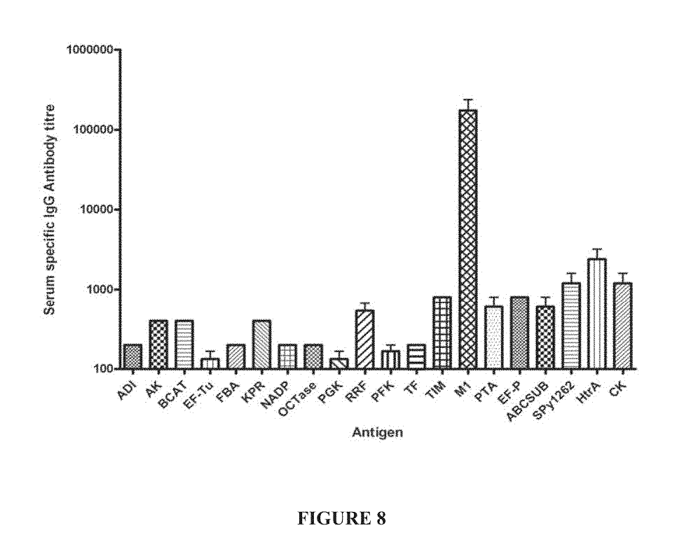

The Aboriginal immune response to vaccine antigens was determined using a pool of serum (n=30) obtained from Aboriginal children living in remote communities of the NT suffering endemic GAS infection. Please see FIG. 8. In comparison to M1 protein, there was a minimal serum antibody response directed against the vaccine antigens in individuals who suffer repeated infection. The Aboriginal population in question suffer repeated GAS infections and high rates of immune sequelae. The lack of an immune response against these set of candidate antigens suggest that these proteins are not involved in triggering autoimmune sequelae. Additionally, the lack of an immune response against these potentially protective antigens, despite repeated infection, suggests that GAS have evolved a mechanism of shielding these antigens from the host immune response.

Percent Identity of Proteins of Interest with Human Proteins

Immunization of humans would be most advantageous if the immunogenic protein does not share homology with human proteins, thus reducing the potential of triggering immune sequelae. For this reason, an essential characteristic of vaccine candidates is ideally no or minimal cross-reactivity of the specific antibodies with human tissue. Advantageously, determining the percent identity between human proteins and the bacterial vaccine candidate proteins is a simple method which may give indications of cross-reactivity.

One of the major GAS vaccine targets is the well-characterised surface M protein. However, the cross-reactivity of anti-M protein antibodies with human tissue has severely hampered the development of M protein based vaccines. Please see Table 8 documenting the percentage amino acid identity with human proteins. It can be seen that the highest percent amino acid identity is 51%, whilst several proteins have no known human homologue.

### Example 5

Opsonisation and/or cell-surface binding assays will be performed. In addition to successful vaccine candidates conferring protection in the intraperitoneal challenge model, an important quality of vaccine candidates is the ability to produce opsonic antibodies (which upon infection promote opsonisation and consequent removal of the pathogen from the host). GAS-surface antibody binding assays will also be performed in order to confirm findings in immunofluorescence microscopy and any opsonisation experiments. These experiments will be extended to all antigens showing protective efficacy.

Testing of other immunization/challenge routes, in addition to the use of other adjuvants, in particular the human approved adjuvant Alum will also be explored for the protective antigens identified in the intraperitoneal challenge experiment.

The possibility of immunizing with a cocktail of protective antigens will also be explored following the above listed experiments. Incorporating antigens found to be protective against multiple challenge routes (intraperitoneal, intravenous, mucosal and subcutaneous) into a cocktail formulation should result in the formulation of a novel GAS vaccine.

### Example 6

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

### REFERENCES

[1] Lindahl, G., M. Stalhammar-Carlemalm, and T. Areschoug, Surface proteins of Streptococcus agalactiae and related proteins in other bacterial pathogens. Clin Microbiol Rev, 2005. 18(1): p. 102-27.
[2] Schrag, S. J., et al., Emergence of Streptococcus pneumoniae with very-high-level resistance to penicillin. Antimicrob Agents Chemother, 2004. 48(8): p. 3016-23.
[3] McCormick, A. W., et al., Geographic diversity and temporal trends of antimicrobial resistance in Streptococcus pneumoniae in the United States. Nat Med, 2003. 9(4): p. 424-30.
[4] Sela, S. and A. Barzilai, Why do we fail with penicillin in the treatment of group A streptococcus infections? Ann Med, 1999. 31(5): p. 303-7.
[5] Cunningham, M. W., Pathogenesis of group A streptococcal infections. Clin Microbiol Rev, 2000. 13 (3): p. 470-511.
[6] Carapetis, J. R., A. C. Steer, E. K. Mulholland, M. Weber. The global burden of group A streptococcal diseases. Lancet Infect Dis, 2005. 5 (11): p. 685-694.
[7] Fischetti, V., Streptococcal M protein: molecular design and biological behaviour. Clin Microbiol Rev, 1989. 2(3): p. 285-314.
[8] Cunningham, M. W., Autoimmunity and molecular mimicry in the pathogenesis of post-streptococcal heart disease. Frontiers in Bioscience, 2003. 8: p. s533-543.
[9] Dale, J. B. and S. T. Shulman, Dynamic epidemiology of group A streptococcal serotypes. Lancet, 2002. 359(9309): p. 889.
[10] Kaplan, E. L., J. T. Wotton, and D. R. Johnson, Dynamic epidemiology of group A streptococcal serotypes associated with pharyngitis. Lancet, 2001. 358(9290): p. 1334-7.
[11] D'Alessandri, R., et al., Protective studies with group A streptococcal M protein vaccine. III. Challenge of volunteers after systemic or intranasal immunization with Type 3 or Type 12 group A Streptococcus. J Infect Dis, 1978. 138(6): p. 712-8.
[12] Fox, E. N., et al., Protective study with a group A streptococcal M protein vaccine. Infectivity challenge of human volunteers. J Clin Invest, 1973. 52(8): p. 1885-92.
[13] Polly, S. M., et al., Protective studies with a group A streptococcal M protein vaccine. II. Challenge of volunteers after local immunization in the upper respiratory tract. J Infect Dis, 1975. 131(3): p. 217-24.
[14] Massed, B. F., L. H. Honikman, and J. Amezcua, Rheumatic fever following streptococcal vaccination. Report of three cases. JAMA, 1969. 207(6): p. 1115-9.
[15] Bessen, D., V. Fischetti, Synthetic peptide vaccine against mucosal colonization by group A streptococci. Protection against a heterologous M serotype with shared C repeat region epitopes. J Immunol, 1990. 145: p. 1251-1256.
[16] Medaglini, D., G. Pozzi, T. King, V. Fischetti, Mucosal and systemic immune responses to a recombinant protein expressed on the surface of the oral commensal bacterium Streptococcus gordonii after oral colonization. Proc. Natl. Acad. Sci. USA 1995. 92: p. 6868-6872.
[17] Beachey, E. H., J. M. Seyer, J. B. Dale, W. A. Simpson, et al., Type-specific protective immunity evoked by synthetic peptide of Streptococcus pyogenes M protein. Nature 1981, 292, 457-459.
[18] Dale, J., J. Seyer, E. Beachey, Type-specific immunogenicity of a chemically synthesized peptide fragment of type 5 streptococcal M protein. J. Exp. Med. 1983, 158, 1727-1732.
[19] Dale, J., E. Chiang, J. Lederer, Recombinant tetravalent group A streptococcal M protein vaccine. J. Immunol. 1993, 151, 2188-2194.
[20] Dale, J. B., M. Simmons, E. C. Chiang, E. Y. Chiang, Recombinant, octavalent group A streptococcal M protein vaccine. Vaccine 1996, 14, 944-948.
[21] Dale, J. B., Multivalent group A streptococcal vaccine designed to optomize the immunogenicity of six tandem M protein fragments. Vaccine 1999, 17, 193-200.
[22] Hall, M. A., S. D. Stroop, M. C. Hu, M. A. Walls, et al., Intranasal immunization with multivalent group A streptococcal vaccines protects mice against intranasal challenge infections. Infect. Immun. 2004, 72, 2507-2512.
[23] Kotloff, K. L., M. Corretti, K. Palmer, J. D. Campbell, et al., Safety and immunogenicity of a recombinant multivalent group A streptococcal vaccine in healthy adults: phase I trial. JAMA 2004, 292, 709-715.
[24] Hu, M. C., M. A. Walls, S. D. Stroop, M. A Reddish, et al., Immunogenicity of a 26-valent group A streptococcal vaccine. Infect. Immun. 2002, 70, 2171-2177.
[25] McNeil, S. A., S. A. Halperin, J. M. Langley, B. Smith, et al., Safety and immunogenicity of 26-valent group A Streptococcus vaccine in healthy adult volunteers. Clin. Infect. Dis. 2005, 41, 1114-1122.
[26] Cole, J. N., et al., Surface analysis and immune reactivity of major cell-wall associated proteins of group A streptococcus. Infect Immun, 2005. 73(5): p. 3137-3146.
[27] Rodreiguez-Ortega, M. J., et al., Characterization and identification of vaccine candidate proteins through analysis of the group A Streptococcus surface proteome. Nat Biotech, 2006. 24(2): p. 191-197.
[28] Ling, E., et al., Glycolytic enzymes associated with the cell surface of Streptococcus pneumoniae are antigenic in humans and elicit protective immune responses in the mouse. Clin Exp Immun, 2004. 138: p. 290-298.

### Tables

TABLE 1
Full protein names, abbreviations and gene names.

| | Abbreviated | Gene |
|---|---|---|
| Protein | Name | Name |
| Arginine deiminase | ADI | sagP |
| 6-phosphofructokinase | PFK | pfkA |
| Fructose-bisphosphate aldolase | FBA | fba |
| Ornithine carbamoyltransferase | OCTase | arcB |
| Triosephosphate isomerase | TIM | tpi |
| Ketopantoate reductase | KPR | apbA |
| Phosphotransacetylase | PTA | SPs0988 |
| Hypothetical protein Spy1262 | Spy1262 | Spy1262 |
| NADP-dependent GAPDH | NADP-GAPDH | gapN |
| Branched-chain-amino-acid | BCAT | bcaT |
| aminotransferase | | |
| Phosphoglycerate kinase | PGK | pgk |
| Carbamate kinase | CK | arcC |
| Trigger factor | TF | ropA |
| Adenylate kinase | AK | adk |
| ABC transporter, substrate | ABC SUB | spyM18_0312 |
| binding protein | | |
| Ribosome recycling factor | RRF | rrf |
| High temperature requirement | HtrA | htrA |
| A serine protease | | |
| Elongation factor P | EF-P | efp |
| Elongation factor Tu | EF-Tu | tufA |

TABLE 2
Sequence type and clinical origin of strains
used in screening experiments.

| Strain | Sequence Type | Clinical origin |
|---|---|---|
| 5448 | emm1 | Invasive, |
| | | STSS and NF |
| NS88.2 | emm98.1 | Invasive blood |
| pM1 | emm1 | NK |
| DSM2071 | emm23 | NK |
| HSC5 | emm5 | Throat isolate |
| | | associated with |
| | | rheumatic fever |
| ALAB49 | emm53 | Impetigo lesion |
| NS192 | emm100 | Renal transplant, |
| | | septic (blood) |
| 20174 | emm3 | Severe invasive |

NK, not known.

TABLE 3
Conservation of protein antigens between sequenced GAS genomes.
BlastP interrogation of published GAS genome sequences.

| | SF370 | 591 | MGAS10394 | MGAS315 | MGAS8232 | SSI-1 | MGAS10270 | MGAS10750 |
|---|---|---|---|---|---|---|---|---|
| Protein | (M1) | (M49) | (M6) | (M3) | (M18) | (M3) | (M2) | (M4) |
| ABC Sub | 99% | 99% | 99% | 99% | 100% | 99% | 99% | 99% |
| ADI | 99% | ND | 99% | 100% | 99% | 100% | 99% | 99% |
| AK | 99% | 99% | 99% | 100% | 99% | 100% | 99% | 100% |
| BCAT | 99% | ND | 99% | 100% | 99% | 100% | 99% | 99% |
| CK | 97% | 97% | 97% | 100% | 97% | 100% | 97% | 99% |
| EF-P | 99% | ND | 99% | 100% | 99% | 100% | 99% | 99% |
| EF-Tu | 99% | 98% | 100% | 100% | 98% | 100% | 100% | 100% |
| FBA | 100% | ND | 99% | 99% | 100% | 99% | 100% | 100% |
| HtrA | 100% | 99% | 99% | 99% | 99% | 99% | 99% | 99% |
| KPR | 99% | 98% | 99% | 99% | 100% | 99% | 100% | 99% |
| NADP-GAPDH | 99% | ND | 99% | 99% | 99% | NF | 99% | 99% |
| OCTase | 99% | 99% | 100% | 100% | 100% | 100% | 99% | 99% |
| PFK | 99% | ND | 100% | 99% | 100% | 99% | 99% | 99% |
| PGK | 99% | 98% | 99% | 100% | 99% | 100% | 99% | 99% |
| PTA | 96% | 98% | 98% | 100% | 98% | 100% | 98% | 98% |
| RRF | 100% | ND | 100% | 100% | 100% | 100% | 99% | 99% |
| Spy1262 | 100% | 99% | 99% | 99% | 99% | 99% | 99% | 99% |
| TF | 99% | 100% | 100% | 100% | 99% | 100% | 99% | 99% |
| TIM | 100% | 100% | 99% | 100% | 99% | 100% | 99% | 99% |

| | MGAS2096 | MGAS5005 | MGAS6180 | Manfredo | MGAS9429 |
|---|---|---|---|---|---|
| Protein | (M12) | (M1) | (M28) | (M5) | (M12) |
| ABC Sub | 99% | 99% | 99% | 99% | 99% |
| ADI | 99% | 99% | 99% | 99% | 99% |
| AK | 99% | 99% | 99% | 99% | 99% |
| BCAT | 99% | 99% | 99% | 99% | 99% |
| CK | 97% | 97% | 97% | 99% | 97% |
| EF-P | 99% | 99% | 92% | 98% | 99% |
| EF-Tu | 100% | 99% | 100% | 99% | 100% |
| FBA | 99% | 100% | 100% | 99% | 99% |
| HtrA | 99% | 100% | 99% | 99% | 100% |
| KPR | 99% | 99% | 100% | 99% | 99% |
| NADP-GAPDH | 99% | 99% | 99% | 99% | 99% |
| OCTase | 99% | 99% | 99% | 99% | 99% |
| PFK | 99% | 99% | 99% | 99% | 99% |
| PGK | 99% | 99% | 99% | 98% | 99% |
| PTA | 98% | 96% | 98% | 98% | 98% |
| RRF | 99% | 99% | 99% | 99% | 99% |
| Spy1262 | 99% | 100% | 99% | 99% | 99% |
| TF | 99% | 99% | 99% | 99% | 99% |
| TIM | 100% | 100% | 99% | 99% | 99% |

ND, sequence not detected in incomplete M49 591 genome sequence; NF gene not found in SSI-1 genome sequence.

TABLE 4
Presence of the genes encoding protein antigens across different group A
streptococcal serotypes. Gene presence determined by PCR amplification.

| | ADI | AK | BCAT | EF-Tu | FBA | KPR | NADP-GAPDH | OCTase | PFK | PGK |
|---|---|---|---|---|---|---|---|---|---|---|
| 5448 | + | + | + | + | + | + | + | + | + | + |
| NS88.2 | + | + | + | + | + | + | + | + | + | + |
| pM1 | + | + | + | + | + | + | + | + | + | + |
| DSM2071 | + | + | + | + | + | + | + | + | + | + |
| HSC5 | + | + | + | + | + | + | + | + | + | + |
| ALAB49 | + | + | + | + | + | + | + | + | + | + |
| NS192 | + | + | + | + | + | + | + | + | + | + |
| 20174 | + | + | + | + | + | + | + | + | + | + |

| | RRF | TF | TIM | PTA | EF-P | ABC SUB | CK | HtrA | Spy1262 |
|---|---|---|---|---|---|---|---|---|---|
| 5448 | + | + | + | + | + | + | + | + | + |
| NS88.2 | + | + | + | + | + | + | + | + | + |
| pM1 | + | + | + | ND | ND | ND | ND | ND | ND |
| DSM2071 | + | + | + | + | + | + | + | + | + |
| HSC5 | + | + | + | + | + | + | + | + | + |
| ALAB49 | + | + | + | + | + | + | + | + | + |
| NS192 | + | + | + | + | + | + | + | + | + |
| 20174 | + | + | + | + | + | + | + | + | + |

+, gene present; −, gene not detected by PCR; ND, not determined.

TABLE 5
The detection of protein antigens in cell-wall extracts. Protein presence determined
by Western blotting mutanolysin cell-wall extracts with specific mouse antiserum
obtained after subcutaneous immunisation with recombinant protein antigens.

| | ADI | AK | BCAT | EF-Tu | FBA | KPR | NADP-GAPDH | OCTase | PFK | PGK |
|---|---|---|---|---|---|---|---|---|---|---|
| 5448 | + | − | + | + | + | + | + | + | + | − |
| NS88.2 | + | − | + | − | + | + | + | + | + | + |
| NS192 | + | + | + | + | + | + | + | + | + | + |
| A20 | + | + | + | + | + | + | + | − | + | + |
| HSC5 | + | + | − | + | + | + | − | + | + | + |
| 20174 | + | + | + | + | + | + | + | + | + | + |
| pM1 | + | + | + | + | + | + | − | + | + | + |

| | RRF | TF | CK | PTA | EF-P | ABC SUB | TIM | Spy1262 | HtrA |
|---|---|---|---|---|---|---|---|---|---|
| 5448 | + | + | + | − | + | − | + | − | + |
| NS88.2 | + | + | + | + | − | − | + | + | − |
| NS192 | + | + | + | + | + | + | + | + | + |
| A20 | + | + | + | + | + | − | + | + | + |
| HSC5 | + | + | + | + | + | + | + | + | + |
| 20174 | + | + | + | + | + | − | + | + | + |
| pM1 | + | + | + | + | + | − | + | + | + |

+, protein detected; −, protein not detected.

TABLE 6
Immunogenic Fragment mapping data in relation
to ADI, TF and KPR. (+) represents a positive
response whilst (−) represents a negative
response.

| | | Post-Immune | Sequence |
|---|---|---|---|
| Peptide | Protein/Spot | Sera | Identifier |
| Number | Sequence | reactivity | (SEQ ID NO) |
| ADI | | | |
| 1 | TAQTPIHVYSEIGKL | − | 1 |
| 2 | TPIHVYSEIGKLKKV | + | 2 |
| 3 | HVYSEIGKLKKVLLH | + | 3 |
| 4 | SEIGKLKKVLLHRPG | + | 4 |
| 5 | GKLKKVLLHRPGKEI | − | 5 |
| 6 | KKVLLHRPGKEIENL | − | 6 |
| 7 | LLHRPGKEIENLMPD | − | 7 |
| 8 | RPGKEIENLMPDYLE | − | 8 |
| 9 | KEIENLMPDYLERLL | + | 9 |
| 10 | ENLMPDYLERLLFDD | + | 10 |
| 11 | MPDYLERLLFDDIPF | + | 11 |
| 12 | YLERLLFDDIPFLED | + | 12 |
| 13 | RLLFDDIPFLEDAQK | + | 13 |
| 14 | FDDIPFLEDAQKEHD | + | 14 |
| 15 | IPFLEDAQKEHDAFA | + | 15 |
| 16 | LEDAQKEHDAFAQAL | + | 16 |
| 17 | AQKEHDAFAQALRDE | + | 17 |
| 18 | EHDAFAQALRDEGIE | + | 18 |
| 19 | AFAQALRDEGIEVLY | + | 19 |
| 20 | QALRDEGIEVLYLET | + | 20 |
| 21 | RDEGIEVLYLETLAA | + | 21 |
| 22 | GIEVLYLETLAAESL | + | 22 |
| 23 | VLYLETLAAESLVTP | − | 23 |
| 24 | LETLAAESLVTPEIR | − | 24 |
| 25 | LAAESLVTPEIREAF | − | 25 |
| 26 | ESLVTPEIREAFIDE | + | 26 |
| 27 | VTPEIREAFIDEYLS | + | 27 |
| 28 | EIREAFIDEYLSEAN | + | 28 |
| 29 | EAFIDEYLSEANIRG | + | 29 |
| 30 | IDEYLSEANIRGRAT | + | 30 |
| 31 | YLSEANIRGRATKKA | − | 3I |
| 32 | EANIRGRATKKAIRE | − | 32 |
| 33 | IRGRATKKAIRELLM | + | 33 |
| 34 | RATKKAIRELLMAIE | + | 34 |
| 35 | KKAIRELLMAIEDNQ | + | 35 |
| 36 | IRELLMAIEDNQELI | + | 36 |
| 37 | LLMAIEDNQELIEKT | + | 37 |
| 38 | AIEDNQELIEKTMAG | − | 38 |
| 39 | DNQELIEKTMAGVQK | − | 39 |
| 40 | ELIEKTMAGVQKSEL | − | 40 |
| 41 | EKTMAGVQKSELPEI | − | 41 |
| 42 | MAGVQKSELPEIPAS | − | 42 |
| 43 | VQKSELPEIPASEKG | + | 43 |
| 44 | SELPEIPASEKGLTD | + | 44 |
| 45 | PEIPASEKGLTDLVE | + | 45 |
| 46 | PASEKGLTDLVESSY | + | 46 |
| 47 | EKGLTDLVESSYPFA | − | 47 |
| 48 | LTDLVESSYPFAIDP | + | 48 |
| 49 | LVESSYPFAIDPMPN | + | 49 |
| 50 | SSYPFAIDPMPNLYF | + | 50 |
| 51 | PFAIDPMPNLYFTRD | − | 51 |
| 52 | IDPMPNLYFTRDPFA | + | 52 |
| 53 | MPNLYFTRDPFATIG | + | 53 |
| 54 | LYFTRDPFATIGTGV | + | 54 |
| 55 | TRDPFATIGTGVSLN | − | 55 |
| 56 | PFATIGTGVSLNHMF | − | 56 |
| 57 | TIGTGVSLNHMFSET | − | 57 |
| 58 | TGVSLNHMFSETRNR | − | 58 |
| 59 | SLNHMFSETRNRETL | − | 59 |
| 60 | HMFSETRNRETLYGK | + | 60 |
| 61 | SETRNRETLYGKYIF | + | 61 |
| 62 | RNRETLYGKYIFTHH | + | 62 |
| 63 | ETLYGKYIFTHHPIY | − | 63 |
| 64 | YGKYIFTHHPIYGGG | − | 64 |
| 65 | YIFTHHPIYGGGKVP | − | 65 |
| 66 | THHPIYGGGKVPMVY | − | 66 |
| 67 | PIYGGGKVPMVYDRN | + | 67 |
| 68 | GGGKVPMVYDRNETT | + | 68 |
| 69 | KVPMVYDRNETTRIE | + | 69 |
| 70 | MVYDRNETTRIEGGD | + | 70 |
| 71 | DRNETTRIEGGDELV | + | 71 |
| 72 | ETTRIEGGDELVLSK | + | 72 |
| 73 | RIEGGDELVLSKDVL | + | 73 |
| 74 | GGDELVLSKDVLAVG | + | 74 |
| 75 | ELVLSKDVLAVGISQ | − | 75 |
| 76 | LSKDVLAVGISQRTD | − | 76 |
| 77 | DVLAVGISQRTDAAS | − | 77 |
| 78 | AVGISQRTDAASIEK | − | 78 |
| 79 | ISQRTDAASIEKLLV | − | 79 |
| 80 | RTDAASIEKLLVNIF | + | 80 |
| 81 | AASIEKLLVNIFKQN | − | 81 |
| 82 | IEKLLVNIFKQNLGF | − | 82 |
| 83 | LLVNIFKQNLGFKKV | − | 83 |
| 84 | NIFKQNLGFKKVLAF | − | 84 |
| 85 | KQNLGFKKVLAFEFA | − | 85 |
| 86 | LGFKKVLAFEFANNR | − | 86 |
| 87 | KKVLAFEFANNRKFM | − | 87 |
| 88 | LAFEFANNRKFMHLD | − | 88 |
| 89 | EFANNRKFMHLDTVF | − | 89 |
| 90 | NNRKFMHLDTVFTMV | − | 90 |
| 91 | KFMHLDTVFTMVDYD | + | 91 |
| 92 | HLDTVFTMVDYDKFT | − | 92 |
| 93 | TVFTMVDYDKFTIHP | + | 93 |
| 94 | TMVDYDKFTIHPEIE | + | 94 |
| 95 | DYDKFTIHPEIEGDL | + | 95 |
| 96 | KFTIHPEIEGDLRVY | + | 96 |
| 97 | IHPEIEGDLRVYSVT | + | 97 |
| 98 | EIEGDLRVYSVTYDN | − | 98 |
| 99 | GDLRVYSVTYDNEEL | − | 99 |
| 100 | RVYSVTYDNEELHIV | − | 100 |
| 101 | SVTYDNEELHIVEEK | − | 101 |
| 102 | YDNEELHIVEEKGDL | − | 102 |
| 103 | EELHIVEEKGDLADL | − | 103 |
| 104 | HIVEEKGDLADLLAA | − | 104 |
| 105 | EEKGDLADLLAANLG | + | 105 |
| 106 | GDLADLLAANLGVEK | + | 106 |
| 107 | ADLLAANLGVEKVDL | + | 107 |
| 108 | LAANLGVEKVDLIRC | − | 108 |
| 109 | NLGVEKVDLIRCGGD | − | 109 |
| 110 | VEKVDLIRCGGDNLV | − | 110 |
| 111 | VDLIRCGGDNLVAAG | − | 111 |
| 112 | IRCGGDNLVAAGREQ | − | 112 |
| 113 | GGDNLVAAGREQWND | + | 113 |
| 114 | NLVAAGREQWNDGSN | + | 114 |
| 115 | AAGREQWNDGSNTLT | + | 115 |
| 116 | REQWNDGSNTLTIAP | + | 116 |
| 117 | WNDGSNTLTIAPGW | − | 117 |
| 118 | GSNTLTIAPGWWY | − | 118 |
| 119 | TLTIAPGVWVYNRN | − | 119 |
| 120 | IAPGWWYNRNTIT | − | 120 |
| 121 | GWWYNRNTITNAI | − | 121 |
| 122 | WYNRNTITNAILES | − | 122 |
| 123 | NRNTITNAILESKGL | − | 123 |
| 124 | TITNAILESKGLKLI | − | 124 |
| 125 | NAILESKGLKLIKIH | − | 125 |
| 126 | LESKGLKLIKIHGSE | − | 126 |
| 127 | KGLKLIKIHGSELVR | − | 127 |
| 128 | KLIKIHGSELVRGRG | − | 128 |
| 129 | KIHGSELVRGRGGPR | − | 129 |
| 130 | GSELVRGRGGPRCMS | − | 130 |
| 131 | LVRGRGGPRCMSMPF | − | 131 |
| 132 | GRGGPRCMSMPFERE | + | 132 |
| 133 | GGPRCMSMPFEREDI | + | 133 |
| KPR | | | |
| 1 | MLVYIAGSGAMGCRF | − | 134 |
| 2 | YIAGSGAMGCRFGYQ | − | 135 |
| 3 | GSGAMGCRFGYQISK | − | 136 |
| 4 | AMGCRFGYQISKTNN | − | 137 |
| 5 | CRFGYQISKTNNDVI | − | 138 |
| 6 | GYQISKTNNDVILLD | + | 139 |
| 7 | ISKTNNDVILLDNWE | + | 140 |
| 8 | TNNDVILLDNWEDHI | + | 141 |
| 9 | DVILLDNWEDHINAI | + | 142 |
| 10 | LLDNWEDHINAIKEN | + | 143 |
| 11 | NWEDHINAIKENGLV | + | 144 |
| 12 | DHINAIKENGLWTG | + | 145 |
| 13 | NAIKENGLWTGDVE | + | 146 |
| 14 | KENGLWTGDVEETV | + | 147 |
| 15 | GLWTGDVEETVKLP | + | 148 |
| 16 | VTGDVEETVKLPIMK | + | 149 |
| 17 | DVEETVKLPIMKPTE | + | 150 |
| 18 | ETVKLPIMKPTEATQ | + | 151 |
| 19 | KLPIMKPTEATQEAD | − | 152 |
| 20 | IMKPTEATQEADLII | − | 153 |
| 21 | PTEATQEADLIILFT | − | 154 |
| 22 | ATQEADLIILFTKAM | − | 155 |
| 23 | EADLIILFTKAMQLP | − | 156 |
| 24 | LIILFTKAMQLPQML | − | 157 |
| 25 | LFTKAMQLPQMLQDI | − | 158 |
| 26 | KAMQLPQMLQDIKGI | − | 159 |
| 27 | QLPQMLQDIKGIIGK | + | 177 |
| 28 | QMLQDIKGIIGKETK | − | 161 |
| 29 | QDIKGIIGKETKVLC | − | 162 |
| 30 | KGIIGKETKVLCLLN | − | 163 |
| 31 | IGKETKVLCLLNGLG | − | 164 |
| 32 | ETKVLCLLNGLGHED | − | 165 |
| 33 | VLCLLNGLGHEDVIR | − | 166 |
| 34 | LLNGLGHEDVIRQYI | − | 167 |
| 35 | GLGHEDVIRQYIPEH | + | 168 |
| 36 | HEDVIRQYIPEHNIL | + | 169 |
| 37 | VIRQYIPEHNILMGV | − | 170 |
| 38 | QYIPEHNILMGVTVW | − | 171 |
| 39 | PEHNILMGVTVWTAG | − | 172 |
| 40 | NILMGVTVWTAGLEG | − | 173 |
| 41 | MGVTVWTAGLEGPGR | − | 174 |
| 42 | TVWTAGLEGPGRAHL | + | 175 |
| 43 | TAGLEGPGRAHLQGV | + | 176 |
| 44 | LEGPGRAHLQGVGAL | + | 178 |
| 45 | PGRAHLQGVGALNLQ | + | 179 |
| 46 | AHLQGVGALNLQSMD | + | 180 |
| 47 | QGVGALNLQSMDPNN | + | 181 |
| 48 | GALNLQSMDPNNQDA | + | 182 |
| 49 | NLQSMDPNNQDAGHQ | + | 183 |
| 50 | SMDPNNQDAGHQVAD | − | 184 |
| 51 | PNNQDAGHQVADLLN | + | 185 |
| 52 | QDAGHQVADLLNKAN | + | 186 |
| 53 | GHQVADLLNKANLNA | + | 187 |
| 54 | VADLLNKANLNATYD | + | 188 |
| 55 | LLNKANLNATYDENV | + | 189 |
| 56 | KANLNATYDENWPN | + | 190 |
| 57 | LNATYDENWPNIWR | + | 191 |
| 58 | TYDENWPNIWRKAC | + | 192 |
| 59 | ENWPNIWRKACVNG | + | 193 |
| 60 | VPNIWRKACVNGTMN | − | 194 |
| 61 | IWRKACVNGTMNSTC | − | 195 |
| 62 | KACVNGTMNSTCALL | − | 196 |
| 63 | VNGTMNSTCALLDCT | − | 197 |
| 64 | TMNSTCALLDCTIGE | − | 198 |
| 65 | STCALLDCTIGELFA | + | 199 |
| 66 | ALLDCTIGELFASED | − | 200 |
| 67 | DCTIGELFASEDGLK | − | 201 |
| 68 | IGELFASEDGLKMVK | − | 202 |
| 69 | LFASEDGLKMVKEII | + | 203 |
| 70 | SEDGLKMVKEIIHEF | + | 204 |
| 71 | GLKMVKEIIHEFVIV | − | 205 |
| 72 | MVKEIIHEFVIVGQA | − | 206 |
| 73 | EIIHEFVIVGQAEGV | − | 207 |
| 74 | HEFVIVGQAEGVELN | − | 208 |
| 75 | VIVGQAEGVELNEEE | − | 209 |
| 76 | GQAEGVELNEEEITQ | + | 210 |
| 77 | EGVELNEEEITQYVM | + | 211 |
| 78 | ELNEEEITQYVMDTS | + | 212 |
| 79 | EEEITQYVMDTSVKA | + | 213 |
| 80 | ITQYVMDTSVKAAHH | + | 214 |
| 81 | YVMDTSVKAAHHYPS | + | 215 |
| 82 | DTSVKAAHHYPSMHQ | + | 216 |
| 83 | VKAAHHYPSMHQDLV | + | 217 |
| 84 | AHHYPSMHQDLVQNH | + | 218 |
| 85 | YPSMHQDLVQNHRLT | + | 219 |
| 86 | MHQDLVQNHRLTEID | + | 220 |
| 87 | DLVQNHRLTEIDFIN | + | 221 |
| 88 | QNHRLTEIDFINGAV | + | 222 |
| 89 | RLTEIDFINGAVNTK | + | 223 |
| 90 | EIDFINGAVNTKGEK | + | 224 |
| 91 | FINGAVNTKGEKLGI | + | 225 |
| 92 | GAVNTKGEKLGINTP | + | 226 |
| 93 | NTKGEKLGINTPYCR | + | 227 |
| 94 | GEKLGINTPYCRMIT | + | 228 |
| 95 | LGINTPYCRMITELV | + | 229 |
| 96 | NTPYCRMITELVHAK | + | 230 |
| 97 | YCRMITELVHAKEAV | + | 231 |
| 98 | MITELVHAKEAVLNI | + | 232 |
| 99 | ITELVHAKEAVLNIQ | + | 233 |
| TF | | | |
| 1 | MSTSFENKATNRGVI | − | 234 |
| 2 | SFENKATNRGVITFT | − | 235 |
| 3 | NKATNRGVITFTISQ | − | 236 |
| 4 | TNRGVITFTISQDKI | − | 237 |
| 5 | GVITFTISQDKIKPA | − | 238 |
| 6 | TFTISQDKIKPALDK | − | 239 |
| 7 | ISQDKIKPALDKAFN | − | 240 |
| 8 | DKIKPALDKAFNKIK | − | 241 |
| 9 | KPALDKAFNKIKKDL | − | 242 |
| 10 | LDKAFNKIKKDLNAP | − | 243 |
| 11 | AFNKIKKDLNAPGFR | − | 244 |
| 12 | KIKKDLNAPGFRKGH | + | 245 |
| 13 | KDLNAPGFRKGHMPR | + | 246 |
| 14 | NAPGFRKGHMPRPVF | + | 247 |
| 15 | GFRKGHMPRPVFNQK | − | 248 |
| 16 | KGHMPRPVFNQKFGE | + | 249 |
| 17 | MPRPVFNQKFGEEVL | + | 250 |
| 18 | PVFNQKFGEEVLYED | + | 251 |
| 19 | NQKFGEEVLYEDALN | + | 252 |
| 20 | FGEEVLYEDALNIVL | + | 253 |
| 21 | EVLYEDALNIVLPEA | + | 254 |
| 22 | YEDALNIVLPEAYEA | + | 255 |
| 23 | ALNIVLPEAYEAAVT | + | 256 |
| 24 | IVLPEAYEAAVTELG | + | 257 |
| 25 | PEAYEAAVTELGLDV | + | 258 |
| 26 | YEAAVTELGLDWAQ | − | 259 |
| 27 | AVTELGLDWAQPKI | − | 260 |
| 28 | ELGLDWAQPKIDW | − | 261 |
| 29 | LDWAQPKIDWSME | − | 262 |
| 30 | VAQPKIDWSMEKGK | − | 263 |
| 31 | PKIDWSMEKGKEWT | − | 264 |
| 32 | DWSMEKGKEWTLSA | − | 265 |
| 33 | SMEKGKEWTLSAEW | − | 266 |
| 34 | KGKEWTLSAEWTKP | − | 267 |
| 35 | EWTLSAEWTKPEVK | − | 268 |
| 36 | LSAEWTKPEVKLGD | − | 269 |
| 37 | EWTKPEVKLGDYKN | + | 270 |
| 38 | TKPEVKLGDYKNLW | + | 271 |
| 39 | EVKLGDYKNLWEVD | + | 272 |
| 40 | LGDYKNLWEVDASK | + | 273 |
| 41 | YKNLWEVDASKEVS | + | 274 |
| 42 | LWEVDASKEVSDED | − | 275 |
| 43 | EVDASKEVSDEDVDA | − | 276 |
| 44 | ASKEVSDEDVDAKIE | − | 277 |
| 45 | EVSDEDVDAKIERER | + | 278 |
| 46 | DEDVDAKIERERQNL | + | 279 |
| 47 | VDAKIERERQNLAEL | + | 280 |
| 48 | KIERERQNLAELIIK | + | 281 |
| 49 | RERQNLAELIIKDGE | + | 282 |
| 50 | QNLAELIIKDGEAAQ | − | 283 |
| 51 | AELIIKDGEAAQGDT | − | 284 |
| 52 | IIKDGEAAQGDTWI | − | 285 |
| 53 | DGEAAQGDTWIDFV | − | 286 |
| 54 | AAQGDTWIDFVGSV | − | 287 |
| 55 | GDTWIDFVGSVDGV | − | 288 |
| 56 | WIDFVGSVDGVEFD | + | 289 |
| 57 | DFVGSVDGVEFDGGK | + | 290 |
| 58 | GSVDGVEFDGGKGDN | + | 291 |
| 59 | DGVEFDGGKGDNFSL | + | 292 |
| 60 | EFDGGKGDNFSLELG | + | 293 |
| 61 | GGKGDNFSLELGSGQ | − | 294 |
| 62 | GDNFSLELGSGQFIP | + | 295 |
| 63 | FSLELGSGQFIPGFE | + | 296 |
| 64 | ELGSGQFIPGFEDQL | + | 297 |
| 65 | SGQFIPGFEDQLVGA | + | 298 |
| 66 | FIPGFEDQLVGAKAG | + | 299 |
| 67 | GFEDQLVGAKAGDEV | − | 300 |
| 68 | DQLVGAKAGDEVEVN | − | 301 |
| 69 | VGAKAGDEVEVNVTF | − | 302 |
| 70 | KAGDEVEVNVTFPES | − | 303 |
| 71 | DEVEVNVTFPESYQA | − | 304 |
| 72 | EVNVTFPESYQAEDL | − | 305 |
| 73 | VTFPESYQAEDLAGK | − | 306 |
| 74 | PESYQAEDLAGKAAK | − | 307 |
| 75 | YQAEDLAGKAAKFMT | − | 308 |
| 76 | EDLAGKAAKFMTTIH | − | 309 |
| 77 | AGKAAKFMTTIHEVK | − | 310 |
| 78 | AAKFMTTIHEVKTKE | − | 311 |
| 79 | FMTTIHEVKTKEVPE | + | 312 |
| 80 | TIHEVKTKEVPELDD | + | 313 |
| 81 | EVKTKEVPELDDELA | − | 314 |
| 82 | TKEVPELDDELAKDI | − | 315 |
| 83 | VPELDDELAKDIDED | + | 316 |
| 84 | LDDELAKDIDEDVDT | + | 317 |
| 85 | ELAKDIDEDVDTLED | + | 318 |
| 86 | KDIDEDVDTLEDLKV | + | 319 |
| 87 | DEDVDTLEDLKVKYR | + | 320 |
| 88 | VDTLEDLKVKYRKEL | + | 321 |
| 89 | LEDLKVKYRKELEAA | + | 322 |
| 90 | LKVKYRKELEAAQET | + | 323 |
| 91 | KYRKELEAAQETAYD | + | 324 |
| 92 | KELEAAQETAYDDAV | − | 325 |
| 93 | EAAQETAYDDAVEGA | − | 326 |
| 94 | QETAYDDAVEGAAIE | + | 327 |
| 95 | AYDDAVEGAAIELAV | − | 328 |
| 96 | DAVEGAAIELAVANA | − | 329 |
| 97 | EGAAIELAVANAEIV | − | 330 |
| 98 | AIELAVANAEIVDLP | − | 331 |
| 99 | LAVANAEIVDLPEEM | − | 332 |
| 100 | ANAEIVDLPEEMIHE | − | 333 |
| 101 | EIVDLPEEMIHEEVN | + | 334 |
| 102 | DLPEEMIHEEVNRSV | + | 335 |
| 103 | EEMIHEEVNRSVNEF | + | 336 |
| 104 | IHEEVNRSVNEFMGN | + | 337 |
| 105 | EVNRSVNEFMGNMQR | + | 338 |
| 106 | RSVNEFMGNMQRQGI | − | 339 |
| 107 | NEFMGNMQRQGISPE | − | 340 |
| 108 | MGNMQRQGISPEMYF | + | 341 |
| 109 | MQRQGISPEMYFQLT | + | 342 |
| 110 | QGISPEMYFQLTGTT | + | 343 |
| 111 | SPEMYFQLTGTTQED | + | 344 |
| 112 | MYFQLTGTTQEDLHN | + | 345 |
| 113 | QLTGTTQEDLHNQYS | − | 346 |
| 114 | GTTQEDLHNQYSAEA | − | 347 |
| 115 | QEDLHNQYSAEADKR | − | 348 |
| 116 | LHNQYSAEADKRVKT | + | 349 |
| 117 | QYSAEADKRVKTNLV | + | 350 |
| 118 | AEADKRVKTNLVIEA | − | 351 |
| 119 | DKRVKTNLVIEAIAK | + | 352 |
| 120 | VKTNLVIEAIAKAEG | + | 353 |
| 121 | NLVIEAIAKAEGFEA | + | 354 |
| 122 | IEAIAKAEGFEATDS | + | 355 |
| 123 | IAKAEGFEATDSEIE | + | 356 |
| 124 | AEGFEATDSEIEQEI | + | 357 |
| 125 | FEATDSEIEQEINDL | + | 358 |
| 126 | TDSEIEQEINDLATE | − | 359 |
| 127 | EIEQEINDLATEYNM | − | 360 |
| 128 | QEINDLATEYNMPAD | + | 361 |
| 129 | NDLATEYNMPADQVR | + | 362 |
| 130 | ATEYNMPADQVRSLL | − | 363 |
| 131 | YNMPADQVRSLLSAD | − | 364 |
| 132 | PADQVRSLLSADMLK | + | 365 |
| 133 | QVRSLLSADMLKHDI | + | 366 |
| 134 | SLLSADMLKHDIAMK | + | 367 |
| 135 | SADMLKHDIAMKKAV | − | 368 |
| 136 | MLKHDIAMKKAVEVI | − | 369 |
| 137 | HDIAMKKAVEVITST | − | 370 |
| 138 | AMKKAVEVITSTASV | − | 371 |
| 139 | MKKAVEVITSTASVK | − | 160 |

TABLE 7
Protective antigens in streptococcal species.

| | Protective in | |
|---|---|---|
| | Group A Streptococcus | Streptococcus |
| Protein | (intraperitoneal challenge results) | pneumoniae |
| Arginine deiminase | ✓ | Not known |
| (ADI) | | |
| Fructose-bis- | x | ✓ |
| phosphate aldolase | | Ling et al. [28] |
| (FBA) | | |
| Ketopantoate | ✓ | Not known |
| reductase (KPR) | | |
| Trigger factor (TF) | ✓ | Not known |

✓ = protective antigen x = non-protective antigen.

TABLE 8
Percent amino acid identity between protein antigens and the human
proteome. BlastP interrogation of human genomic databases.

| | % identity to |
|---|---|
| Protein | human protein |
| ADI | NS |
| PFK | 37% |
| PGK | 43% |
| TIM | 40% |
| NADP-GAPDH | 33% |
| FBA | NS |
| KPR | NS |
| BCAT | 32% |
| AK | 39% |
| OCTase | 40% |
| EF-Tu | 51% |
| RRF | 28% |
| TF | NS |
| PTA | NS |
| EF-P | NS |
| ABC-Sub | NS |
| Spy1262 | NS |
| CK | NS |
| HtrA | 8% |

NS, no significant homology.

TABLE 9
Databank entry details for proteins used in this study

| | | Genbank | |
|---|---|---|---|
| | Genbank Nucleotide | Protein | SwissProt |
| Protein | Accession No | Accession No | Accession No |
| ABC Sub | AE009977 Directs to | AAL97071 | Q8P2K8 |
| | MGAS8232 AE009977 | | |
| ADI | AF468045 | AAM22954 | Q8K5F0 |
| AK | AE014137 Directs to | AAM78668 | P69882 |
| | MGAS315 AE014074 | | |
| BCAT | AE014149 Directs to | AAM79233 | Q8K7U5 |
| | MGAS315 AE014074 | | |
| CK | AE014159 Directs to | AAM79798 | Q8K6Q9 |
| | MGAS315 AE014074 | | |
| EF-P | AE014166 Directs to | AAM80181 | P68774 |
| | MGAS315 AE014074 | | |
| EF-Tu | AE014145 Directs to | AAM79039 | Q8K872 |
| | MGAS315 AE014074 | | |
| FBA | AE006614 Directs to | AAK34600 | P68905 |
| | M1 (SF370) AE004092 | | |
| HtrA | NP_270119.1 Directs to | AAK34840 | A2RH30 |
| | M1 (SF370) AE004092 | | |
| KPR | AE010020 Directs to | AAL97561 | Q8P1F1 |
| | MGAS8232 AE010020 | | |
| NADP- | AE014157 Directs to | AAM79652 | Q8K707 |
| GAPDH | MGAS315 AE014074 | | |
| OCTase | AE014159 Directs to | AAM79801 | P65609 |
| | MGAS315 AE014074 | | |
| PFK | AE010047 Directs to | AAL97841 | Q8P0S6 |
| | MGAS8232 AE010047 | | |
| PGK | AE014167 Directs to | AAM80231 | Q8K5W7 |
| | MGAS315 AE014074 | | |
| PTA | BA000034 | BAC64083 | Q878S0 |
| | (SSI-1 complete genome) | | |
| RRF | AE009989 Directs to | AAL97224 | Q8P274 |
| | MGAS8232 AE009989 | | |
| Spy1262 | AE006565 Directs to | AAK34116 | Q99ZE5 |
| | M1 (SF370) AE004092 | | |
| TF | AE014167 Directs to | AAM80241 | Q879L7 |
| | MGAS315 AE014074 | | |
| TIM | AE006516 Directs to | AAK33587 | P69887 |
| | M1 (SF370) AE004092 | | |

1. A method of eliciting an immune response in an animal, including the step of administering a pharmaceutical composition comprising one or more isolated immunogenic fragments of an isolated protein consisting of amino acids 1-218 of SEQ ID NO:373 or a variant having an amino acid sequence at least 90% amino acid sequence identity thereto, wherein said isolated protein lacks significant sequence identity to a human protein and/or does not elicit a specific immune response in a human following natural infection with S. pyogenes, wherein each of the one or more isolated immunogenic fragments comprises an amino acid sequence consisting of at least 15 contiguous amino acids selected from amino acids 1-218 of SEQ ID NO:373 or from the amino acid sequence of said variant, to thereby elicit the immune response in said animal.

2. The method of claim 1, wherein the selected amino acids comprise between 15 and 50 contiguous amino acids of the isolated protein or said variant.

3. The method of claim 1, wherein at least one of the one or more isolated immunogenic fragments comprise an amino acid sequence selected from the group consisting of SEQ ID NOS:1 to 69.

4. The method of claim 1, wherein said variant has an amino acid sequence having at least 95% sequence identity with amino acids 1-218 of SEQ ID NO:373.

5. The method of claim 1, wherein the immune response is or comprises a humoral immune response.

6. The method of claim 1, wherein the pharmaceutical composition further comprises a pharmaceutically acceptable carrier, diluent or excipient.


SEQUENCE LISTING (373 sequences)

SEQ ID NO: 1 - protein, 15 aa, Streptococcus pyogenes
TAQTPIHVYSEIGKL

SEQ ID NO: 2 - protein, 15 aa, Streptococcus pyogenes
TPIHVYSEIGKLKKV

SEQ ID NO: 3 - protein, 15 aa, Streptococcus pyogenes
HVYSEIGKLKKVLLH

SEQ ID NO: 4 - protein, 15 aa, Streptococcus pyogenes
SEIGKLKKVLLHRPG

SEQ ID NO: 5 - protein, 15 aa, Streptococcus pyogenes
GKLKKVLLHRPGKEI

SEQ ID NO: 6 - protein, 15 aa, Streptococcus pyogenes
KKVLLHRPGKEIENL

SEQ ID NO: 7 - protein, 15 aa, Streptococcus pyogenes
LLHRPGKEIENLMPD

SEQ ID NO: 8 - protein, 15 aa, Streptococcus pyogenes
RPGKEIENLMPDYLE

SEQ ID NO: 9 - protein, 15 aa, Streptococcus pyogenes
KEIENLMPDYLERLL

SEQ ID NO: 10 - protein, 15 aa, Streptococcus pyogenes
ENLMPDYLERLLFDD

SEQ ID NO: 11 - protein, 15 aa, Streptococcus pyogenes
MPDYLERLLFDDIPF

SEQ ID NO: 12 - protein, 15 aa, Streptococcus pyogenes
YLERLLFDDIPFLED

SEQ ID NO: 13 - protein, 15 aa, Streptococcus pyogenes
RLLFDDIPFLEDAQK

SEQ ID NO: 14 - protein, 15 aa, Streptococcus pyogenes
FDDIPFLEDAQKEHD

SEQ ID NO: 15 - protein, 15 aa, Streptococcus pyogenes
IPFLEDAQKEHDAFA

SEQ ID NO: 16 - protein, 15 aa, Streptococcus pyogenes
LEDAQKEHDAFAQAL

SEQ ID NO: 17 - protein, 15 aa, Streptococcus pyogenes
AQKEHDAFAQALRDE

SEQ ID NO: 18 - protein, 15 aa, Streptococcus pyogenes
EHDAFAQALRDEGIE

SEQ ID NO: 19 - protein, 15 aa, Streptococcus pyogenes
AFAQALRDEGIEVLY

SEQ ID NO: 20 - protein, 15 aa, Streptococcus pyogenes
QALRDEGIEVLYLET

SEQ ID NO: 21 - protein, 15 aa, Streptococcus pyogenes
RDEGIEVLYLETLAA

SEQ ID NO: 22 - protein, 15 aa, Streptococcus pyogenes
GIEVLYLETLAAESL

SEQ ID NO: 23 - protein, 15 aa, Streptococcus pyogenes
VLYLETLAAESLVTP

SEQ ID NO: 24 - protein, 15 aa, Streptococcus pyogenes
LETLAAESLVTPEIR

SEQ ID NO: 25 - protein, 15 aa, Streptococcus pyogenes
LAAESLVTPEIREAF

SEQ ID NO: 26 - protein, 15 aa, Streptococcus pyogenes
ESLVTPEIREAFIDE

SEQ ID NO: 27 - protein, 15 aa, Streptococcus pyogenes
VTPEIREAFIDEYLS

SEQ ID NO: 28 - protein, 15 aa, Streptococcus pyogenes
EIREAFIDEYLSEAN

SEQ ID NO: 29 - protein, 15 aa, Streptococcus pyogenes
EAFIDEYLSEANIRG

SEQ ID NO: 30 - protein, 15 aa, Streptococcus pyogenes
IDEYLSEANIRGRAT

SEQ ID NO: 31 - protein, 15 aa, Streptococcus pyogenes
YLSEANIRGRATKKA

SEQ ID NO: 32 - protein, 15 aa, Streptococcus pyogenes
EANIRGRATKKAIRE

SEQ ID NO: 33 - protein, 15 aa, Streptococcus pyogenes
IRGRATKKAIRELLM

SEQ ID NO: 34 - protein, 15 aa, Streptococcus pyogenes
RATKKAIRELLMAIE

SEQ ID NO: 35 - protein, 15 aa, Streptococcus pyogenes
KKAIRELLMAIEDNQ

SEQ ID NO: 36 - protein, 15 aa, Streptococcus pyogenes
IRELLMAIEDNQELI

SEQ ID NO: 37 - protein, 15 aa, Streptococcus pyogenes
LLMAIEDNQELIEKT

SEQ ID NO: 38 - protein, 15 aa, Streptococcus pyogenes
AIEDNQELIEKTMAG

SEQ ID NO: 39 - protein, 15 aa, Streptococcus pyogenes
DNQELIEKTMAGVQK

SEQ ID NO: 40 - protein, 15 aa, Streptococcus pyogenes
ELIEKTMAGVQKSEL

SEQ ID NO: 41 - protein, 15 aa, Streptococcus pyogenes
EKTMAGVQKSELPEI

SEQ ID NO: 42 - protein, 15 aa, Streptococcus pyogenes
MAGVQKSELPEIPAS

SEQ ID NO: 43 - protein, 15 aa, Streptococcus pyogenes
VQKSELPEIPASEKG

SEQ ID NO: 44 - protein, 15 aa, Streptococcus pyogenes
SELPEIPASEKGLTD

SEQ ID NO: 45 - protein, 15 aa, Streptococcus pyogenes
PEIPASEKGLTDLVE

SEQ ID NO: 46 - protein, 15 aa, Streptococcus pyogenes
PASEKGLTDLVESSY

SEQ ID NO: 47 - protein, 15 aa, Streptococcus pyogenes
EKGLTDLVESSYPFA

SEQ ID NO: 48 - protein, 15 aa, Streptococcus pyogenes
LTDLVESSYPFAIDP

SEQ ID NO: 49 - protein, 15 aa, Streptococcus pyogenes
LVESSYPFAIDPMPN

SEQ ID NO: 50 - protein, 15 aa, Streptococcus pyogenes
SSYPFAIDPMPNLYF

SEQ ID NO: 51 - protein, 15 aa, Streptococcus pyogenes
PFAIDPMPNLYFTRD

SEQ ID NO: 52 - protein, 15 aa, Streptococcus pyogenes
IDPMPNLYFTRDPFA

SEQ ID NO: 53 - protein, 15 aa, Streptococcus pyogenes
MPNLYFTRDPFATIG

SEQ ID NO: 54 - protein, 15 aa, Streptococcus pyogenes
LYFTRDPFATIGTGV

SEQ ID NO: 55 - protein, 15 aa, Streptococcus pyogenes
TRDPFATIGTGVSLN

SEQ ID NO: 56 - protein, 15 aa, Streptococcus pyogenes
PFATIGTGVSLNHMF

SEQ ID NO: 57 - protein, 15 aa, Streptococcus pyogenes
TIGTGVSLNHMFSET

SEQ ID NO: 58 - protein, 15 aa, Streptococcus pyogenes
TGVSLNHMFSETRNR

SEQ ID NO: 59 - protein, 15 aa, Streptococcus pyogenes
SLNHMFSETRNRETL

SEQ ID NO: 60 - protein, 15 aa, Streptococcus pyogenes
HMFSETRNRETLYGK

SEQ ID NO: 61 - protein, 15 aa, Streptococcus pyogenes
SETRNRETLYGKYIF

SEQ ID NO: 62 - protein, 15 aa, Streptococcus pyogenes
RNRETLYGKYIFTHH

SEQ ID NO: 63 - protein, 15 aa, Streptococcus pyogenes
ETLYGKYIFTHHPIY

SEQ ID NO: 64 - protein, 15 aa, Streptococcus pyogenes
YGKYIFTHHPIYGGG

SEQ ID NO: 65 - protein, 15 aa, Streptococcus pyogenes
YIFTHHPIYGGGKVP

SEQ ID NO: 66 - protein, 15 aa, Streptococcus pyogenes
THHPIYGGGKVPMVY

SEQ ID NO: 67 - protein, 15 aa, Streptococcus pyogenes
PIYGGGKVPMVYDRN

SEQ ID NO: 68 - protein, 15 aa, Streptococcus pyogenes
GGGKVPMVYDRNETT

SEQ ID NO: 69 - protein, 15 aa, Streptococcus pyogenes
KVPMVYDRNETTRIE

SEQ ID NO: 70 - protein, 15 aa, Streptococcus pyogenes
MVYDRNETTRIEGGD

SEQ ID NO: 71 - protein, 15 aa, Streptococcus pyogenes
DRNETTRIEGGDELV

SEQ ID NO: 72 - protein, 15 aa, Streptococcus pyogenes
ETTRIEGGDELVLSK

SEQ ID NO: 73 - protein, 15 aa, Streptococcus pyogenes
RIEGGDELVLSKDVL

SEQ ID NO: 74 - protein, 15 aa, Streptococcus pyogenes
GGDELVLSKDVLAVG

SEQ ID NO: 75 - protein, 15 aa, Streptococcus pyogenes
ELVLSKDVLAVGISQ

SEQ ID NO: 76 - protein, 15 aa, Streptococcus pyogenes
LSKDVLAVGISQRTD

SEQ ID NO: 77 - protein, 15 aa, Streptococcus pyogenes
DVLAVGISQRTDAAS

SEQ ID NO: 78 - protein, 15 aa, Streptococcus pyogenes
AVGISQRTDAASIEK

SEQ ID NO: 79 - protein, 15 aa, Streptococcus pyogenes
ISQRTDAASIEKLLV

SEQ ID NO: 80 - protein, 15 aa, Streptococcus pyogenes
RTDAASIEKLLVNIF

SEQ ID NO: 81 - protein, 15 aa, Streptococcus pyogenes
AASIEKLLVNIFKQN

SEQ ID NO: 82 - protein, 15 aa, Streptococcus pyogenes
IEKLLVNIFKQNLGF

SEQ ID NO: 83 - protein, 15 aa, Streptococcus pyogenes
LLVNIFKQNLGFKKV

SEQ ID NO: 84 - protein, 15 aa, Streptococcus pyogenes
NIFKQNLGFKKVLAF

SEQ ID NO: 85 - protein, 15 aa, Streptococcus pyogenes
KQNLGFKKVLAFEFA

SEQ ID NO: 86 - protein, 15 aa, Streptococcus pyogenes
LGFKKVLAFEFANNR

SEQ ID NO: 87 - protein, 15 aa, Streptococcus pyogenes
KKVLAFEFANNRKFM

SEQ ID NO: 88 - protein, 15 aa, Streptococcus pyogenes
LAFEFANNRKFMHLD

SEQ ID NO: 89 - protein, 15 aa, Streptococcus pyogenes
EFANNRKFMHLDTVF

SEQ ID NO: 90 - protein, 15 aa, Streptococcus pyogenes
NNRKFMHLDTVFTMV

SEQ ID NO: 91 - protein, 15 aa, Streptococcus pyogenes
KFMHLDTVFTMVDYD

SEQ ID NO: 92 - protein, 15 aa, Streptococcus pyogenes
HLDTVFTMVDYDKFT

SEQ ID NO: 93 - protein, 15 aa, Streptococcus pyogenes
TVFTMVDYDKFTIHP

SEQ ID NO: 94 - protein, 15 aa, Streptococcus pyogenes
TMVDYDKFTIHPEIE

SEQ ID NO: 95 - protein, 15 aa, Streptococcus pyogenes
DYDKFTIHPEIEGDL

SEQ ID NO: 96 - protein, 15 aa, Streptococcus pyogenes
KFTIHPEIEGDLRVY

SEQ ID NO: 97 - protein, 15 aa, Streptococcus pyogenes
IHPEIEGDLRVYSVT

SEQ ID NO: 98 - protein, 15 aa, Streptococcus pyogenes
EIEGDLRVYSVTYDN

SEQ ID NO: 99 - protein, 15 aa, Streptococcus pyogenes
GDLRVYSVTYDNEEL

SEQ ID NO: 100 - protein, 15 aa, Streptococcus pyogenes
RVYSVTYDNEELHIV

SEQ ID NO: 101 - protein, 15 aa, Streptococcus pyogenes
SVTYDNEELHIVEEK

SEQ ID NO: 102 - protein, 15 aa, Streptococcus pyogenes
YDNEELHIVEEKGDL

SEQ ID NO: 103 - protein, 15 aa, Streptococcus pyogenes
EELHIVEEKGDLADL

SEQ ID NO: 104 - protein, 15 aa, Streptococcus pyogenes
HIVEEKGDLADLLAA

SEQ ID NO: 105 - protein, 15 aa, Streptococcus pyogenes
EEKGDLADLLAANLG

SEQ ID NO: 106 - protein, 15 aa, Streptococcus pyogenes
GDLADLLAANLGVEK

SEQ ID NO: 107 - protein, 15 aa, Streptococcus pyogenes
ADLLAANLGVEKVDL

SEQ ID NO: 108 - protein, 15 aa, Streptococcus pyogenes
LAANLGVEKVDLIRC

SEQ ID NO: 109 - protein, 15 aa, Streptococcus pyogenes
NLGVEKVDLIRCGGD

SEQ ID NO: 110 - protein, 15 aa, Streptococcus pyogenes
VEKVDLIRCGGDNLV

SEQ ID NO: 111 - protein, 15 aa, Streptococcus pyogenes
VDLIRCGGDNLVAAG

SEQ ID NO: 112 - protein, 15 aa, Streptococcus pyogenes
IRCGGDNLVAAGREQ

SEQ ID NO: 113 - protein, 15 aa, Streptococcus pyogenes
GGDNLVAAGREQWND

SEQ ID NO: 114 - protein, 15 aa, Streptococcus pyogenes
NLVAAGREQWNDGSN

SEQ ID NO: 115 - protein, 15 aa, Streptococcus pyogenes
AAGREQWNDGSNTLT

SEQ ID NO: 116 - protein, 15 aa, Streptococcus pyogenes
REQWNDGSNTLTIAP

SEQ ID NO: 117 - protein, 14 aa, Streptococcus pyogenes
WNDGSNTLTIAPGW

SEQ ID NO: 118 - protein, 13 aa, Streptococcus pyogenes
GSNTLTIAPGWWY

SEQ ID NO: 119 - protein, 14 aa, Streptococcus pyogenes
TLTIAPGVWVYNRN

SEQ ID NO: 120 - protein, 13 aa, Streptococcus pyogenes
IAPGWWYNRNTIT

SEQ ID NO: 121 - protein, 13 aa, Streptococcus pyogenes
GWWYNRNTITNAI

SEQ ID NO: 122 - protein, 14 aa, Streptococcus pyogenes
WYNRNTITNAILES

SEQ ID NO: 123 - protein, 15 aa, Streptococcus pyogenes
NRNTITNAILESKGL

SEQ ID NO: 124 - protein, 15 aa, Streptococcus pyogenes
TITNAILESKGLKLI

SEQ ID NO: 125 - protein, 15 aa, Streptococcus pyogenes
NAILESKGLKLIKIH

SEQ ID NO: 126 - protein, 15 aa, Streptococcus pyogenes
LESKGLKLIKIHGSE

SEQ ID NO: 127 - protein, 15 aa, Streptococcus pyogenes
KGLKLIKIHGSELVR

SEQ ID NO: 128 - protein, 15 aa, Streptococcus pyogenes
KLIKIHGSELVRGRG

SEQ ID NO: 129 - protein, 15 aa, Streptococcus pyogenes
KIHGSELVRGRGGPR

SEQ ID NO: 130 - protein, 15 aa, Streptococcus pyogenes
GSELVRGRGGPRCMS

SEQ ID NO: 131 - protein, 15 aa, Streptococcus pyogenes
LVRGRGGPRCMSMPF

SEQ ID NO: 132 - protein, 15 aa, Streptococcus pyogenes
GRGGPRCMSMPFERE

SEQ ID NO: 133 - protein, 15 aa, Streptococcus pyogenes
GGPRCMSMPFEREDI

SEQ ID NO: 134 - protein, 15 aa, Streptococcus pyogenes
MLVYIAGSGAMGCRF

SEQ ID NO: 135 - protein, 15 aa, Streptococcus pyogenes
YIAGSGAMGCRFGYQ

SEQ ID NO: 136 - protein, 15 aa, Streptococcus pyogenes
GSGAMGCRFGYQISK

SEQ ID NO: 137 - protein, 15 aa, Streptococcus pyogenes
AMGCRFGYQISKTNN

SEQ ID NO: 138 - protein, 15 aa, Streptococcus pyogenes
CRFGYQISKTNNDVI

SEQ ID NO: 139 - protein, 15 aa, Streptococcus pyogenes
GYQISKTNNDVILLD

SEQ ID NO: 140 - protein, 15 aa, Streptococcus pyogenes
ISKTNNDVILLDNWE

SEQ ID NO: 141 - protein, 15 aa, Streptococcus pyogenes
TNNDVILLDNWEDHI

SEQ ID NO: 142 - protein, 15 aa, Streptococcus pyogenes
DVILLDNWEDHINAI

SEQ ID NO: 143 - protein, 15 aa, Streptococcus pyogenes
LLDNWEDHINAIKEN

SEQ ID NO: 144 - protein, 15 aa, Streptococcus pyogenes
NWEDHINAIKENGLV

SEQ ID NO: 145 - protein, 14 aa, Streptococcus pyogenes
DHINAIKENGLWTG

SEQ ID NO: 146 - protein, 14 aa, Streptococcus pyogenes
NAIKENGLWTGDVE

SEQ ID NO: 147 - protein, 14 aa, Streptococcus pyogenes
KENGLWTGDVEETV

SEQ ID NO: 148 - protein, 14 aa, Streptococcus pyogenes
GLWTGDVEETVKLP

SEQ ID NO: 149 - protein, 15 aa, Streptococcus pyogenes
VTGDVEETVKLPIMK

SEQ ID NO: 150 - protein, 15 aa, Streptococcus pyogenes
DVEETVKLPIMKPTE

SEQ ID NO: 151 - protein, 15 aa, Streptococcus pyogenes
ETVKLPIMKPTEATQ

SEQ ID NO: 152 - protein, 15 aa, Streptococcus pyogenes
KLPIMKPTEATQEAD

SEQ ID NO: 153 - protein, 15 aa, Streptococcus pyogenes
IMKPTEATQEADLII

SEQ ID NO: 154 - protein, 15 aa, Streptococcus pyogenes
PTEATQEADLIILFT

SEQ ID NO: 155 - protein, 15 aa, Streptococcus pyogenes
ATQEADLIILFTKAM

SEQ ID NO: 156 - protein, 15 aa, Streptococcus pyogenes
EADLIILFTKAMQLP

SEQ ID NO: 157 - protein, 15 aa, Streptococcus pyogenes
LIILFTKAMQLPQML

SEQ ID NO: 158 - protein, 15 aa, Streptococcus pyogenes
LFTKAMQLPQMLQDI

SEQ ID NO: 159 - protein, 15 aa, Streptococcus pyogenes
KAMQLPQMLQDIKGI

SEQ ID NO: 160 - protein, 15 aa, Streptococcus pyogenes
QLPQMLQDIKGIIGK

SEQ ID NO: 161 - protein, 15 aa, Streptococcus pyogenes
QMLQDIKGIIGKETK

SEQ ID NO: 162 - protein, 15 aa, Streptococcus pyogenes
QDIKGIIGKETKVLC

SEQ ID NO: 163 - protein, 15 aa, Streptococcus pyogenes
KGIIGKETKVLCLLN

SEQ ID NO: 164 - protein, 15 aa, Streptococcus pyogenes
IGKETKVLCLLNGLG

SEQ ID NO: 165 - protein, 15 aa, Streptococcus pyogenes
ETKVLCLLNGLGHED

SEQ ID NO: 166 - protein, 15 aa, Streptococcus pyogenes
VLCLLNGLGHEDVIR

SEQ ID NO: 167 - protein, 15 aa, Streptococcus pyogenes
LLNGLGHEDVIRQYI

SEQ ID NO: 168 - protein, 15 aa, Streptococcus pyogenes
GLGHEDVIRQYIPEH

SEQ ID NO: 169 - protein, 15 aa, Streptococcus pyogenes
HEDVIRQYIPEHNIL

SEQ ID NO: 170 - protein, 15 aa, Streptococcus pyogenes
VIRQYIPEHNILMGV

SEQ ID NO: 171 - protein, 15 aa, Streptococcus pyogenes
QYIPEHNILMGVTVW

SEQ ID NO: 172 - protein, 15 aa, Streptococcus pyogenes
PEHNILMGVTVWTAG

SEQ ID NO: 173 - protein, 15 aa, Streptococcus pyogenes
NILMGVTVWTAGLEG

SEQ ID NO: 174 - protein, 15 aa, Streptococcus pyogenes
MGVTVWTAGLEGPGR

SEQ ID NO: 175 - protein, 15 aa, Streptococcus pyogenes
TVWTAGLEGPGRAHL

SEQ ID NO: 176 - protein, 15 aa, Streptococcus pyogenes
TAGLEGPGRAHLQGV

SEQ ID NO: 177 - skipped

SEQ ID NO: 178 - protein, 15 aa, Streptococcus pyogenes
LEGPGRAHLQGVGAL

SEQ ID NO: 179 - protein, 15 aa, Streptococcus pyogenes
PGRAHLQGVGALNLQ

SEQ ID NO: 180 - protein, 15 aa, Streptococcus pyogenes
AHLQGVGALNLQSMD

SEQ ID NO: 181 - protein, 15 aa, Streptococcus pyogenes
QGVGALNLQSMDPNN

SEQ ID NO: 182 - protein, 15 aa, Streptococcus pyogenes
GALNLQSMDPNNQDA

SEQ ID NO: 183 - protein, 15 aa, Streptococcus pyogenes
NLQSMDPNNQDAGHQ

SEQ ID NO: 184 - protein, 15 aa, Streptococcus pyogenes
SMDPNNQDAGHQVAD

SEQ ID NO: 185 - protein, 15 aa, Streptococcus pyogenes
PNNQDAGHQVADLLN

SEQ ID NO: 186 - protein, 15 aa, Streptococcus pyogenes
QDAGHQVADLLNKAN

SEQ ID NO: 187 - protein, 15 aa, Streptococcus pyogenes
GHQVADLLNKANLNA

SEQ ID NO: 188 - protein, 15 aa, Streptococcus pyogenes
VADLLNKANLNATYD

SEQ ID NO: 189 - protein, 15 aa, Streptococcus pyogenes
LLNKANLNATYDENV

SEQ ID NO: 190 - protein, 14 aa, Streptococcus pyogenes
KANLNATYDENWPN

SEQ ID NO: 191 - protein, 14 aa, Streptococcus pyogenes
LNATYDENWPNIWR

SEQ ID NO: 192 - protein, 14 aa, Streptococcus pyogenes
TYDENWPNIWRKAC

SEQ ID NO: 193 - protein, 14 aa, Streptococcus pyogenes
ENWPNIWRKACVNG

SEQ ID NO: 194 - protein, 15 aa, Streptococcus pyogenes
VPNIWRKACVNGTMN

SEQ ID NO: 195 - protein, 15 aa, Streptococcus pyogenes
IWRKACVNGTMNSTC

SEQ ID NO: 196 - protein, 15 aa, Streptococcus pyogenes
KACVNGTMNSTCALL

SEQ ID NO: 197 - protein, 15 aa, Streptococcus pyogenes
VNGTMNSTCALLDCT

SEQ ID NO: 198 - protein, 15 aa, Streptococcus pyogenes
TMNSTCALLDCTIGE

SEQ ID NO: 199 - protein, 15 aa, Streptococcus pyogenes
STCALLDCTIGELFA

SEQ ID NO: 200 - protein, 15 aa, Streptococcus pyogenes
ALLDCTIGELFASED

SEQ ID NO: 201 - protein, 15 aa, Streptococcus pyogenes
DCTIGELFASEDGLK

SEQ ID NO: 202 - protein, 15 aa, Streptococcus pyogenes
IGELFASEDGLKMVK

SEQ ID NO: 203 - protein, 15 aa, Streptococcus pyogenes
LFASEDGLKMVKEII

SEQ ID NO: 204 - protein, 15 aa, Streptococcus pyogenes
SEDGLKMVKEIIHEF

SEQ ID NO: 205 - protein, 15 aa, Streptococcus pyogenes
GLKMVKEIIHEFVIV

SEQ ID NO: 206 - protein, 15 aa, Streptococcus pyogenes
MVKEIIHEFVIVGQA

SEQ ID NO: 207 - protein, 15 aa, Streptococcus pyogenes
EIIHEFVIVGQAEGV

SEQ ID NO: 208 - protein, 15 aa, Streptococcus pyogenes
HEFVIVGQAEGVELN

SEQ ID NO: 209 - protein, 15 aa, Streptococcus pyogenes
VIVGQAEGVELNEEE

SEQ ID NO: 210 - protein, 15 aa, Streptococcus pyogenes
GQAEGVELNEEEITQ

SEQ ID NO: 211 - protein, 15 aa, Streptococcus pyogenes
EGVELNEEEITQYVM

SEQ ID NO: 212 - protein, 15 aa, Streptococcus pyogenes
ELNEEEITQYVMDTS

SEQ ID NO: 213 - protein, 15 aa, Streptococcus pyogenes
EEEITQYVMDTSVKA

SEQ ID NO: 214 - protein, 15 aa, Streptococcus pyogenes
ITQYVMDTSVKAAHH

SEQ ID NO: 215 - protein, 15 aa, Streptococcus pyogenes
YVMDTSVKAAHHYPS

SEQ ID NO: 216 - protein, 15 aa, Streptococcus pyogenes
DTSVKAAHHYPSMHQ

SEQ ID NO: 217 - protein, 15 aa, Streptococcus pyogenes
VKAAHHYPSMHQDLV

SEQ ID NO: 218 - protein, 15 aa, Streptococcus pyogenes
AHHYPSMHQDLVQNH

SEQ ID NO: 219 - protein, 15 aa, Streptococcus pyogenes
YPSMHQDLVQNHRLT

SEQ ID NO: 220 - protein, 15 aa, Streptococcus pyogenes
MHQDLVQNHRLTEID

SEQ ID NO: 221 - protein, 15 aa, Streptococcus pyogenes
DLVQNHRLTEIDFIN

SEQ ID NO: 222 - protein, 15 aa, Streptococcus pyogenes
QNHRLTEIDFINGAV

SEQ ID NO: 223 - protein, 15 aa, Streptococcus pyogenes
RLTEIDFINGAVNTK

SEQ ID NO: 224 - protein, 15 aa, Streptococcus pyogenes
EIDFINGAVNTKGEK

SEQ ID NO: 225 - protein, 15 aa, Streptococcus pyogenes
FINGAVNTKGEKLGI

SEQ ID NO: 226 - protein, 15 aa, Streptococcus pyogenes
GAVNTKGEKLGINTP

SEQ ID NO: 227 - protein, 15 aa, Streptococcus pyogenes
NTKGEKLGINTPYCR

SEQ ID NO: 228 - protein, 15 aa, Streptococcus pyogenes
GEKLGINTPYCRMIT

SEQ ID NO: 229 - protein, 15 aa, Streptococcus pyogenes
LGINTPYCRMITELV

SEQ ID NO: 230 - protein, 15 aa, Streptococcus pyogenes
NTPYCRMITELVHAK

SEQ ID NO: 231 - protein, 15 aa, Streptococcus pyogenes
YCRMITELVHAKEAV

SEQ ID NO: 232 - protein, 15 aa, Streptococcus pyogenes
MITELVHAKEAVLNI

SEQ ID NO: 233 - protein, 15 aa, Streptococcus pyogenes
ITELVHAKEAVLNIQ

SEQ ID NO: 234 - protein, 15 aa, Streptococcus pyogenes
MSTSFENKATNRGVI

SEQ ID NO: 235 - protein, 15 aa, Streptococcus pyogenes
SFENKATNRGVITFT

SEQ ID NO: 236 - protein, 15 aa, Streptococcus pyogenes
NKATNRGVITFTISQ

SEQ ID NO: 237 - protein, 15 aa, Streptococcus pyogenes
TNRGVITFTISQDKI

SEQ ID NO: 238 - protein, 15 aa, Streptococcus pyogenes
GVITFTISQDKIKPA

SEQ ID NO: 239 - protein, 15 aa, Streptococcus pyogenes
TFTISQDKIKPALDK

SEQ ID NO: 240 - protein, 15 aa, Streptococcus pyogenes
ISQDKIKPALDKAFN

SEQ ID NO: 241 - protein, 15 aa, Streptococcus pyogenes
DKIKPALDKAFNKIK

SEQ ID NO: 242 - protein, 15 aa, Streptococcus pyogenes
KPALDKAFNKIKKDL

SEQ ID NO: 243 - protein, 15 aa, Streptococcus pyogenes
LDKAFNKIKKDLNAP

SEQ ID NO: 244 - protein, 15 aa, Streptococcus pyogenes
AFNKIKKDLNAPGFR

SEQ ID NO: 245 - protein, 15 aa, Streptococcus pyogenes
KIKKDLNAPGFRKGH

SEQ ID NO: 246 - protein, 15 aa, Streptococcus pyogenes
KDLNAPGFRKGHMPR

SEQ ID NO: 247 - protein, 15 aa, Streptococcus pyogenes
NAPGFRKGHMPRPVF

SEQ ID NO: 248 - protein, 15 aa, Streptococcus pyogenes
GFRKGHMPRPVFNQK

SEQ ID NO: 249 - protein, 15 aa, Streptococcus pyogenes
KGHMPRPVFNQKFGE

SEQ ID NO: 250 - protein, 15 aa, Streptococcus pyogenes
MPRPVFNQKFGEEVL

SEQ ID NO: 251 - protein, 15 aa, Streptococcus pyogenes
PVFNQKFGEEVLYED

SEQ ID NO: 252 - protein, 15 aa, Streptococcus pyogenes
NQKFGEEVLYEDALN

SEQ ID NO: 253 - protein, 15 aa, Streptococcus pyogenes
FGEEVLYEDALNIVL

SEQ ID NO: 254 - protein, 15 aa, Streptococcus pyogenes
EVLYEDALNIVLPEA

SEQ ID NO: 255 - protein, 15 aa, Streptococcus pyogenes
YEDALNIVLPEAYEA

SEQ ID NO: 256 - protein, 15 aa, Streptococcus pyogenes
ALNIVLPEAYEAAVT

SEQ ID NO: 257 - protein, 15 aa, Streptococcus pyogenes
IVLPEAYEAAVTELG

SEQ ID NO: 258 - protein, 15 aa, Streptococcus pyogenes
PEAYEAAVTELGLDV

SEQ ID NO: 259 - protein, 14 aa, Streptococcus pyogenes
YEAAVTELGLDWAQ

SEQ ID NO: 260 - protein, 14 aa, Streptococcus pyogenes
AVTELGLDWAQPKI

SEQ ID NO: 261 - protein, 13 aa, Streptococcus pyogenes
ELGLDWAQPKIDW

SEQ ID NO: 262 - protein, 13 aa, Streptococcus pyogenes
LDWAQPKIDWSME

SEQ ID NO: 263 - protein, 14 aa, Streptococcus pyogenes
VAQPKIDWSMEKGK

SEQ ID NO: 264 - protein, 14 aa, Streptococcus pyogenes
PKIDWSMEKGKEWT

SEQ ID NO: 265 - protein, 14 aa, Streptococcus pyogenes
DWSMEKGKEWTLSA

SEQ ID NO: 266 - protein, 14 aa, Streptococcus pyogenes
SMEKGKEWTLSAEW

SEQ ID NO: 267 - protein, 14 aa, Streptococcus pyogenes
KGKEWTLSAEWTKP

SEQ ID NO: 268 - protein, 14 aa, Streptococcus pyogenes
EWTLSAEWTKPEVK

SEQ ID NO: 269 - protein, 14 aa, Streptococcus pyogenes
LSAEWTKPEVKLGD

SEQ ID NO: 270 - protein, 14 aa, Streptococcus pyogenes
EWTKPEVKLGDYKN

SEQ ID NO: 271 - protein, 14 aa, Streptococcus pyogenes
TKPEVKLGDYKNLW

SEQ ID NO: 272 - protein, 14 aa, Streptococcus pyogenes
EVKLGDYKNLWEVD

SEQ ID NO: 273 - protein, 14 aa, Streptococcus pyogenes
LGDYKNLWEVDASK

SEQ ID NO: 274 - protein, 14 aa, Streptococcus pyogenes
YKNLWEVDASKEVS

SEQ ID NO: 275 - protein, 14 aa, Streptococcus pyogenes
LWEVDASKEVSDED

SEQ ID NO: 276 - protein, 15 aa, Streptococcus pyogenes
EVDASKEVSDEDVDA

SEQ ID NO: 277 - protein, 15 aa, Streptococcus pyogenes
ASKEVSDEDVDAKIE

SEQ ID NO: 278 - protein, 15 aa, Streptococcus pyogenes
EVSDEDVDAKIERER

SEQ ID NO: 279 - protein, 15 aa, Streptococcus pyogenes
DEDVDAKIERERQNL

SEQ ID NO: 280 - protein, 15 aa, Streptococcus pyogenes
VDAKIERERQNLAEL

SEQ ID NO: 281 - protein, 15 aa, Streptococcus pyogenes
KIERERQNLAELIIK

SEQ ID NO: 282 - protein, 15 aa, Streptococcus pyogenes
RERQNLAELIIKDGE

SEQ ID NO: 283 - protein, 15 aa, Streptococcus pyogenes
QNLAELIIKDGEAAQ

SEQ ID NO: 284 - protein, 15 aa, Streptococcus pyogenes
AELIIKDGEAAQGDT

SEQ ID NO: 285 - protein, 12 aa, Streptococcus pyogenes
KDGEAAQGDTWI

SEQ ID NO: 286 - protein, 14 aa, Streptococcus pyogenes
DGEAAQGDTWIDFV

SEQ ID NO: 287 - protein, 14 aa, Streptococcus pyogenes
AAQGDTWIDFVGSV

SEQ ID NO: 288 - protein, 14 aa, Streptococcus pyogenes
GDTWIDFVGSVDGV

SEQ ID NO: 289 - protein, 14 aa, Streptococcus pyogenes
WIDFVGSVDGVEFD

SEQ ID NO: 290 - protein, 15 aa, Streptococcus pyogenes
DFVGSVDGVEFDGGK

SEQ ID NO: 291 - protein, 15 aa, Streptococcus pyogenes
GSVDGVEFDGGKGDN

SEQ ID NO: 292 - protein, 15 aa, Streptococcus pyogenes
DGVEFDGGKGDNFSL

SEQ ID NO: 293 - protein, 15 aa, Streptococcus pyogenes
EFDGGKGDNFSLELG

SEQ ID NO: 294 - protein, 15 aa, Streptococcus pyogenes
GGKGDNFSLELGSGQ

SEQ ID NO: 295 - protein, 15 aa, Streptococcus pyogenes
GDNFSLELGSGQFIP

SEQ ID NO: 296 - protein, 15 aa, Streptococcus pyogenes
FSLELGSGQFIPGFE

SEQ ID NO: 297 - protein, 15 aa, Streptococcus pyogenes
ELGSGQFIPGFEDQL

SEQ ID NO: 298 - protein, 15 aa, Streptococcus pyogenes
SGQFIPGFEDQLVGA

SEQ ID NO: 299 - protein, 15 aa, Streptococcus pyogenes
FIPGFEDQLVGAKAG

SEQ ID NO: 300 - protein, 15 aa, Streptococcus pyogenes
GFEDQLVGAKAGDEV

SEQ ID NO: 301 - protein, 15 aa, Streptococcus pyogenes
DQLVGAKAGDEVEVN

SEQ ID NO: 302 - protein, 15 aa, Streptococcus pyogenes
VGAKAGDEVEVNVTF

SEQ ID NO: 303 - protein, 15 aa, Streptococcus pyogenes
KAGDEVEVNVTFPES

SEQ ID NO: 304 - protein, 15 aa, Streptococcus pyogenes
DEVEVNVTFPESYQA

SEQ ID NO: 305 - protein, 15 aa, Streptococcus pyogenes
EVNVTFPESYQAEDL

SEQ ID NO: 306 - protein, 15 aa, Streptococcus pyogenes
VTFPESYQAEDLAGK

SEQ ID NO: 307 - protein, 15 aa, Streptococcus pyogenes
PESYQAEDLAGKAAK

SEQ ID NO: 308 - protein, 15 aa, Streptococcus pyogenes
YQAEDLAGKAAKFMT

SEQ ID NO: 309 - protein, 15 aa, Streptococcus pyogenes
EDLAGKAAKFMTTIH

SEQ ID NO: 310 - protein, 15 aa, Streptococcus pyogenes
AGKAAKFMTTIHEVK

SEQ ID NO: 311 - protein, 15 aa, Streptococcus pyogenes
AAKFMTTIHEVKTKE

SEQ ID NO: 312 - protein, 15 aa, Streptococcus pyogenes
FMTTIHEVKTKEVPE

SEQ ID NO: 313 - protein, 15 aa, Streptococcus pyogenes
TIHEVKTKEVPELDD

SEQ ID NO: 314 - protein, 15 aa, Streptococcus pyogenes
EVKTKEVPELDDELA

SEQ ID NO: 315 - protein, 15 aa, Streptococcus pyogenes
TKEVPELDDELAKDI

SEQ ID NO: 316 - protein, 15 aa, Streptococcus pyogenes
VPELDDELAKDIDED

SEQ ID NO: 317 - protein, 15 aa, Streptococcus pyogenes
LDDELAKDIDEDVDT

SEQ ID NO: 318 - protein, 15 aa, Streptococcus pyogenes
ELAKDIDEDVDTLED

SEQ ID NO: 319 - protein, 15 aa, Streptococcus pyogenes
KDIDEDVDTLEDLKV

SEQ ID NO: 320 - protein, 15 aa, Streptococcus pyogenes
DEDVDTLEDLKVKYR

SEQ ID NO: 321 - protein, 15 aa, Streptococcus pyogenes
VDTLEDLKVKYRKEL

SEQ ID NO: 322 - protein, 15 aa, Streptococcus pyogenes
LEDLKVKYRKELEAA

SEQ ID NO: 323 - protein, 15 aa, Streptococcus pyogenes
LKVKYRKELEAAQET

SEQ ID NO: 324 - protein, 15 aa, Streptococcus pyogenes
KYRKELEAAQETAYD

SEQ ID NO: 325 - protein, 15 aa, Streptococcus pyogenes
KELEAAQETAYDDAV

SEQ ID NO: 326 - protein, 15 aa, Streptococcus pyogenes
EAAQETAYDDAVEGA

SEQ ID NO: 327 - protein, 15 aa, Streptococcus pyogenes
QETAYDDAVEGAAIE

SEQ ID NO: 328 - protein, 15 aa, Streptococcus pyogenes
AYDDAVEGAAIELAV

SEQ ID NO: 329 - protein, 15 aa, Streptococcus pyogenes
DAVEGAAIELAVANA

SEQ ID NO: 330 - protein, 15 aa, Streptococcus pyogenes
EGAAIELAVANAEIV

SEQ ID NO: 331 - protein, 15 aa, Streptococcus pyogenes
AIELAVANAEIVDLP

SEQ ID NO: 332 - protein, 15 aa, Streptococcus pyogenes
LAVANAEIVDLPEEM

SEQ ID NO: 333 - protein, 15 aa, Streptococcus pyogenes
ANAEIVDLPEEMIHE

SEQ ID NO: 334 - protein, 15 aa, Streptococcus pyogenes
EIVDLPEEMIHEEVN

SEQ ID NO: 335 - protein, 15 aa, Streptococcus pyogenes
DLPEEMIHEEVNRSV

SEQ ID NO: 336 - protein, 15 aa, Streptococcus pyogenes
EEMIHEEVNRSVNEF

SEQ ID NO: 337 - protein, 15 aa, Streptococcus pyogenes
IHEEVNRSVNEFMGN

SEQ ID NO: 338 - protein, 15 aa, Streptococcus pyogenes
EVNRSVNEFMGNMQR

SEQ ID NO: 339 - protein, 15 aa, Streptococcus pyogenes
RSVNEFMGNMQRQGI

SEQ ID NO: 340 - protein, 15 aa, Streptococcus pyogenes
NEFMGNMQRQGISPE

SEQ ID NO: 341 - protein, 15 aa, Streptococcus pyogenes
MGNMQRQGISPEMYF

SEQ ID NO: 342 - protein, 15 aa, Streptococcus pyogenes
MQRQGISPEMYFQLT

SEQ ID NO: 343 - protein, 15 aa, Streptococcus pyogenes
QGISPEMYFQLTGTT

SEQ ID NO: 344 - protein, 15 aa, Streptococcus pyogenes
SPEMYFQLTGTTQED

SEQ ID NO: 345 - protein, 15 aa, Streptococcus pyogenes
MYFQLTGTTQEDLHN

SEQ ID NO: 346 - protein, 15 aa, Streptococcus pyogenes
QLTGTTQEDLHNQYS

SEQ ID NO: 347 - protein, 15 aa, Streptococcus pyogenes
GTTQEDLHNQYSAEA

SEQ ID NO: 348 - protein, 15 aa, Streptococcus pyogenes
QEDLHNQYSAEADKR

SEQ ID NO: 349 - protein, 15 aa, Streptococcus pyogenes
LHNQYSAEADKRVKT

SEQ ID NO: 350 - protein, 15 aa, Streptococcus pyogenes
QYSAEADKRVKTNLV

SEQ ID NO: 351 - protein, 15 aa, Streptococcus pyogenes
AEADKRVKTNLVIEA

SEQ ID NO: 352 - protein, 15 aa, Streptococcus pyogenes
DKRVKTNLVIEAIAK

SEQ ID NO: 353 - protein, 15 aa, Streptococcus pyogenes
VKTNLVIEAIAKAEG

SEQ ID NO: 354 - protein, 15 aa, Streptococcus pyogenes
NLVIEAIAKAEGFEA

SEQ ID NO: 355 - protein, 15 aa, Streptococcus pyogenes
IEAIAKAEGFEATDS

SEQ ID NO: 356 - protein, 15 aa, Streptococcus pyogenes
IAKAEGFEATDSEIE

SEQ ID NO: 357 - protein, 15 aa, Streptococcus pyogenes
AEGFEATDSEIEQEI

SEQ ID NO: 358 - protein, 15 aa, Streptococcus pyogenes
FEATDSEIEQEINDL

SEQ ID NO: 359 - protein, 15 aa, Streptococcus pyogenes
TDSEIEQEINDLATE

SEQ ID NO: 360 - protein, 15 aa, Streptococcus pyogenes
EIEQEINDLATEYNM

SEQ ID NO: 361 - protein, 15 aa, Streptococcus pyogenes
QEINDLATEYNMPAD

SEQ ID NO: 362 - protein, 15 aa, Streptococcus pyogenes
NDLATEYNMPADQVR

SEQ ID NO: 363 - protein, 15 aa, Streptococcus pyogenes
ATEYNMPADQVRSLL

SEQ ID NO: 364 - protein, 15 aa, Streptococcus pyogenes
YNMPADQVRSLLSAD

SEQ ID NO: 365 - protein, 15 aa, Streptococcus pyogenes
PADQVRSLLSADMLK

SEQ ID NO: 366 - protein, 15 aa, Streptococcus pyogenes
QVRSLLSADMLKHDI

SEQ ID NO: 367 - protein, 15 aa, Streptococcus pyogenes
SLLSADMLKHDIAMK

SEQ ID NO: 368 - protein, 15 aa, Streptococcus pyogenes
SADMLKHDIAMKKAV

SEQ ID NO: 369 - protein, 15 aa, Streptococcus pyogenes
MLKHDIAMKKAVEVI

SEQ ID NO: 370 - protein, 15 aa, Streptococcus pyogenes
HDIAMKKAVEVITST

SEQ ID NO: 371 - protein, 15 aa, Streptococcus pyogenes
AMKKAVEVITSTASV

SEQ ID NO: 372 - protein, 15 aa, Streptococcus pyogenes
MKKAVEVITSTASVK

SEQ ID NO: 373 - protein, 410 aa, Streptococcus pyogenes
TAQTPIHVYSEIGKLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEKTMAGVQKSELPEIPASEKGLTDLVESSYPFAIDPMPNLYFTRDPFATIGTGVSLNHMFSETRNRETLYGKYIFTHHPIYGGGKVPMVYDRNETTRIEGGDELVLSKDVLAVGISQRTDAASIEKLLVNIFKQNLGFKKVLAFEFANNRKFMHLDTVFTMVDYDKFTIHPEIEGDLRVYSVTYDNEELHIVEEKGDLADLLAANLGVEKVDLIRCGGDNLVAAGREQWNDGSNTLTIAPGVVVVYNRNTITNAILESKGLKLIKIHGSELVRGRGGPRCMSMPFEREDI